(19) United States
(12) Patent Application Publication
(10) Pub. No.: US 2005/0031592 A1
(43) Pub. Date: Feb. 10, 2005

(54) Methods and compositions for inducing immune responses and protective immunity by priming with alpha virus replicon vaccines

(76) Inventors: Denise L. Doolan, Rockville, MD (US); Gary L. Brice, McKees Rock, PA (US); Carlota Dobano-Lazaro, Barcelom (ES); Jeffrey D. Chulay, Chapel Hill, NC (US); Kurt I. Kamrud, Apex, NC (US); Jonathan F. Smith, Cary, NC (US)

Correspondence Address: NAVAL MEDICAL RESEARCH CENTER;ATTN: (CODE 00L), 503 ROBERT GRANT AVENUE, SILVER SPRING, MD, 20910-7500, US

(21) Appl. No.: 10/706,088

(22) Filed: Nov. 13, 2003

Related U.S. Application Data
(60) Provisional application No. 60/425,720, filed on Nov. 13, 2002.

(51) Int. Cl. A61K 48/00; A61K 39/002

(52) U.S. Cl. 424/93.2; 424/191.1

(57) ABSTRACT

The inventive subject matter relates to an immunogenic composition and method of immunizing a subject against malarial disease comprising administering to the subject a priming immunization preparation comprising alphavirus replicons expressing a gene encoding a malarial antigen or combination of malarial antigens and subsequently administering to the subject a boosting immunization preparation comprising the malarial antigen(s) or an expression system containing the antigen(s). The inventive composition and methods result in a robust induction of humoral and cellular immune system responses, including CD8⁺, CD4⁺ and antibody responses.

### FIELD OF THE INVENTION

The inventive subject matter relates to an immunogenic composition and method of immunizing a subject against malarial disease comprising administering a priming immunization preparation containing an alphavirus replicon expressing a gene encoding a malarial antigen or combination of antigens and subsequently administering to the subject a boosting immunization preparation containing the malarial antigen(s) or antigen expression system containing the antigen(s).

### BACKGROUND OF THE INVENTION

Malaria poses an enormous burden on public health throughout the world and occurs in more than 90 countries, inhabited by a total of some 2.4 billion people or 40% of the world's population. Worldwide incidence of the disease is estimated to be on the order of 300-500 million new infections and 2-4 million deaths annually. Mortality due to malaria is estimated to be in the range of 1.5 to 2.7 million deaths annually according to the World Health Organization (WHO). In addition, tens of millions of travelers from North America, Europe, Japan or Australia visit areas of the world with malaria every year. Of these, 10,000-30,000 contract malaria annually. Furthermore, in every military campaign of the past century mounted in areas where malaria was endemic, U.S. forces have suffered more casualties to malaria than from hostile fire, and entire divisions have been rendered non-operative. Finally, in sub-Saharan Africa, it is estimated that annually 1%-4% of gross domestic product (GDP), a minimum of $12 billion, is lost due to malaria (See: Gallup J L, et al. The economic burden of malaria. Am J Trop Med Hyg. 2001 January;64(1-2 Suppl):85-96).

The cost of physical intervention methods intended to interfere with the transmission of the malarial disease, such as bednets and window screens, is often prohibitive and such measures are not highly effective. The availability and cost of prophylactic drugs precludes their use by many of individuals who need them the most. Moreover, the emergence of drug-resistant parasites means that many of the prophylactic drugs that were effective in the past are no longer useful, and many of the newer generation drugs are associated with rare but significant side effects, such as fatal heart rhythms, fatal skin disease, neurological disturbances, and gastrointestinal distress. The increase in insecticide-resistance of the vectors that transmit malaria and the undesirable environmental impact of those insecticides shown to be most effective means that chemical interventions are frequently not useful in combating the disease. These factors emphasize the urgent need for the development of an effective malaria vaccine.

The current status of malaria vaccine development and clinical trials have been the subject of a number of recent reviews (See: Graves P, et al. Vaccines for preventing malaria. Cochrane Database Syst Rev. 2003;(1):CD000129; Moore S A, et al. Malaria vaccines: where are we and where are we going? Lancet Infect Dis. 2002 December;2(12):737-43: Carvalho L J, et al. Malaria vaccine: candidate antigens, mechanisms, constraints and prospects. Scand J. Immunol. 2002 October;56(4):327-43; Greenwood B, et al. Malaria vaccine trials. Chem Immunol. 2002;80:366-95; and Richie T L, et al. Progress and challenges for malaria vaccines. Nature. 2002 Feb. 7;415(6872):694-701). Over the past 15-20 years, a series of Phase 1 and 2 vaccine trials have been reported using synthetic peptides or recombinant proteins based on malarial antigens. Approximately 40 trials were reported by 1998 (See: Engers H D, et al. Malaria vaccine development: current status. Parasitology Today 21998. 14, 56-64). Most of these trials have been directed against sporozoites or liver stages where the use of experimental mosquito challenges allows rapid progress through Phase 1 to Phase 2a preliminary efficacy studies. Anti-sporozoite vaccines tested have included completely synthetic peptides, conjugates of synthetic peptide with proteins such as tetanus toxoid to provide T cell help, recombinant malaria proteins, particle-forming recombinant chimeric constructs, recombinant viruses and bacteria, and DNA vaccines. Several trials of asexual blood stage vaccines have used either synthetic peptide conjugates or recombinant proteins and there has been a single trial of a transmission blocking vaccine (recombinant Pfs25).

A recurring theme in these trials has been the difficulty of obtaining a sufficiently strong and long lasting immune response in humans even though the same vaccine preparation is often strongly immunogenic in test animals. Various strategies seek to overcome this limitation including the exploration of potent immune-stimulatory conjugates or adjuvants to boost the human response or the development of novel vaccine technology platforms and application either alone or in combination with other technologies. The former approach is best illustrated by vaccines directed against the circumsporozoite protein (CSP), the prinicpal sporozotie coat protein. Early studies with recombinant proteins, peptide conjugates, and recombinant protein conjugates were able to elicit anti-CSP antibodies but provided marginal protection in Phase 2a studies and no protection in field studies. More recently, a chimeric protein consisting of a fusion between the CSP and the hepatitis B surface antigen (which forms typical HBsAg particles when expressed in the presence of unmodified HBsAg (the RTS,S vaccine)) has been extensively evaluated in animals and in clinical studies with experimental challenge and field exposure (See: Bojang K A, et al., RTS, S Malaria Vaccine Trial Team. Efficacy of RTS,S/AS02 malaria vaccine against Plasmodium falciparum infection in semi-immune adult men in The Gambia: a randomised trial. Lancet. 2001 Dec. 8;358(9297):1927-34). Although much more immunogenic than recombinant CS protein, the RTS,S is still suboptimal with regard to its ability to protect against malaria. For example, although the RTS,S/ASO2 vaccine could protect 40-50% of volunteers experimentally challenged 2-3 weeks after their last immunization, only one of five volunteers was protected when rechallenged 6 months after the last immunization (See: Stoute J A, et al. Long-term efficacy and immune responses following immunization with the RTS,S malaria vaccine. J Infect Dis. 1998 October;178(4):1139-44). In field studies, vaccine efficacy (end-point defined as “time to first infection”) was 71% (95% CI 46% to 85%) during the first 9 weeks of surveillance but subsequently declined to 0% (95% CI −52% to 34%) in the last 6 weeks (See: Bojang, K. A., et al., RTS, S Malaria Vaccine Trial Team, Efficacy of RTS,S/AS02 malaria vaccine against Plasmodium falciparum infection in semi-immune adult men in The Gambia: a randomised trial, Lancet. 2001 Dec. 8;358(9297): 1927-34). Although considerable efforts are still being directed at the development of protein-based vaccines, alternative technologies such as DNA and viral based vaccines show some promise with regard to immunogenicity and protective efficacy, at least in animal models. Additionally, these molecular based vaccines may prove particularly amenable to multivalent formulations and are probably less expensive to produce, store, and deliver as compared with the more conventional vaccines.

In the malaria model, the capacity of DNA vaccines encoding Plasmodium antigens to induce CD8⁺ CTL and IFN-γ responses and protection against sporozoite challenge in mice (See: Sedegah M, et al. Protection against malaria by immunization with plasmid DNA encoding circumsporozoite protein. Proc Natl Acad Sci USA. 1994 Oct. 11;91(21):9866-70; and Doolan D L, et al. Circumventing genetic restriction of protection against malaria with multigene DNA immunization: CD8+cell-, interferon gamma-, and nitric oxide-dependent immunity. J Exp Med. 1996 Apr. 1;183(4):1739-46) and monkeys has been established (See: Wang R, et al. Induction of antigen-specific cytotoxic T lymphocytes in humans by a malaria DNA vaccine. Science. 1998a Oct 16;282(5388):476-80; Rogers WO, et al. Protection of rhesus macaques against lethal Plasmodium knowlesi malaria by a heterologous DNA priming and poxvirus boosting immunization regimen. Infect Immun. 2002 August;70(8):4329-35; and Rogers WO, et al. Multistage multiantigen heterologous prime boost vaccine for Plasmodium knowlesi malaria provides partial protection in rhesus macaques. Infect Immun. 2001 September;69(9):5565-72). In addition, Phase I and 2a clinical trials have established the safety, tolerability and immunogenicity of DNA vaccines encoding malaria antigens in normal healthy humans (See: Wang R, et al. Simultaneous induction of multiple antigen-specific cytotoxic T lymphocytes in nonhuman primates by immunization with a mixture of four Plasmodium falciparum DNA plasmids. Infect Immun. 1998b Sep;66(9):4193-202, 2000; Le TP, et al. Safety, tolerability and humoral immune responses after intramuscular administration of a malaria DNA vaccine to healthy adult volunteers. Vaccine. 2000 Mar. 17;18(18):1893-901; and Epstein J E, et al. Safety, tolerability, and lack of antibody responses after administration of a PfCSP DNA malaria vaccine via needle or needle-free jet injection, and comparison of intramuscular and combination intramuscular/intradermal routes. Hum Gene Ther. 2002 Sep. 1;13(13):1551-60). However, the immunogenicity of first- and second-generation DNA vaccines in nonhuman primates and in humans has been suboptimal. Even in murine models, DNA vaccines are not effective at activating all arms of the immune system. For example, immunization of mice with plasmid DNA encoding the pre-erythrocytic stage Plasmodium yoelii antigens, PyCSP and PyHEP17 induces antigen-specific cell mediated immune responses and antibody responses and confers sterile protection against sporozoite challenge. However, this protection does not withstand high challenge doses and is not sustained for long periods (M. Sedegah, unpublished; D. L. Doolan, unpublished), and is genetically restricted (See: Doolan, 1996, supra). In the protected mouse strains, PyCSP DNA induces good CD8⁺ CTL responses and good antibody responses, but poor CD4⁺ T cell responses (See Sedegah 1994, supra; and Sedegah M, et al. Boosting with recombinant vaccinia increases immunogenicity and protective efficacy of malaria DNA vaccine. Proc Natl Acad Sci USA. 1998 Jun. 23;95(13):7648-53). PyHEP17 induces poor CD⁸+ CTL and CD4⁺ T cell responses and negligible antibody responses (See Doolan, 1996, supra). It is now generally accepted that although DNA immunization is effective at inducing antigen-specific cellular responses, DNA immunization induces only moderate levels of immune activation (See: Zavala F, et al. A striking property of recombinant poxviruses: efficient inducers of in vivo expansion of primed CD8(+) T cells. Virology. 2001 Feb. 15;280(2):155-9; and Pardoll D M. Spinning molecular immunology into successful immunotherapy. Nat Rev Immunol. 2002 April;2(4):227-38).

Considerable efforts have been directed at evaluating potential immune enhancement strategies for DNA vaccination. Studies in a number of model systems have now established that the immunogenicity and protective efficacy of DNA vaccines may be significantly enhanced by heterologous prime/boost regimens, using vector systems such as recombinant poxviruses or adenoviruses (reviewed in: McShane H, Prime-boost immunization strategies for infectious diseases. Curr Opin Mol Ther. 2002 February;4(1):23-7; Newman M J. Heterologous prime-boost vaccination strategies for HIV-1: augmenting cellular immune responses. Curr Opin Investig Drugs. 2002 March;3(3):374-8; and Hill A V, et al. DNA-based vaccines for malaria: a heterologous prime-boost immunisation strategy. Dev Biol (Basel). 2000;104:171-9). However, the complexity of such recombinant technologies and immunization strategies detracts from some of the major advantages of DNA vaccine technology as compared with more conventional vaccine delivery systems, namely ease of construction, stability and lack of requirement for a cold-chain. In addition, there are safety concerns with using live, attenuated viral vectors. Finally, pre-existing immunity to recombinant viral vectors may limit the boosting potential of recombinant virus immunization and may preclude repeated use of these immunization strategies for different vaccines, and pre-existing immunity to the vector may decrease the effectiveness of recombinant viruses immunizations.

Alphaviruses have successfully been used as viral-based gene delivery vectors. These systems induce transient, high-level antigen expression and have a broad tissue host range. Alphavirus vector systems also have the ability to infect both dividing and non-dividing cells, including antigen-presenting cells, and can induce host immuno-stimulatory responses.

Alphaviruses belong to the Togaviridae family (arbovirus) and are arthropod borne. The virion is spherical and enveloped (60-70 nm diameter), and contains two envelope glycoproteins (E1, E2) as well as an icosahedral capsid protein (28-35 nm). The genome consists of linear single-stranded, positive-sense RNA (12 Kb, 49s RNA, Mol.Wt. 4 million). The infectious genes for nonstructural proteins are located at the 5′ end, which is capped; the 3′ end is polyadenylated. There are two functional segments within the genome: the 5′⅔ encodes self-assembling replicase (enzymatic non-structural proteins) that synthesizes (−) RNA genome, (+) RNA genome, and sub-genomic mRNA; the 3′⅓ (subgenomic mRNA) encodes structural proteins. Each segment contains an independent promoter. Alphavirus replicons are nucleic acids derived from the full-length virus in which the genes encoding the structural proteins (capsid and envelope proteins) have been removed, rendering the replicon capable of replicating within a cell but propagation-incompetent. In alphavirus replicon expression systems, one or more genes encoding the antigen(s) of interest can be inserted after the subgenomic promoter (26S mRNA). When such a replicon is delivered as a DNA molecule to a host cell, the alphaviral replicase machinery encoded on the replicon produces a large quantity of mRNA encoding the desired antigen, and the transfected cell undergoes apoptosis and is taken up by dendritic cells, leading to enhanced antigen presentation (Ying 1999).

Alternatively, the replicon can be delivered as a naked RNA molecule, or most preferably as an alphavirus replicon particle, in which the replicon is packaged in a membrane or lipid vesicle containing the alphavirus structural proteins. Alphavirus replicons are considered propagation-defective “suicide” vectors since they infect antigen-presenting cells and induce apoptosis, but are unable to revert to an infectious state. Their predilection for infecting antigen-presenting cells, including dendritic cells, and their inability to revert to an infectious state makes them very attractive and safe vaccines. Because transfected cells are destroyed and not allowed to produce antigen chronically, theoretical concerns about tolerance, autoimmunity, and integration of plasmid sequences into the host's genome are reduced and alphavirus replicons are considered to have a better safety profile than plasmid DNA vaccines.

Several alphaviruses are being developed as vector delivery systems: Sindbis virus (SIN), Semliki Forest virus (SFV), and Venezuelan equine encephalitis virus (VEE). The present invention involves an attenuated non-propagating Venezuelan equine encephalitis (VEE) replicon vector system that has been developed to express heterologous antigens at high levels while remaining propagation defective. The vector component of the system consists of VEE replicon particles (VRP). VRP contain a VEE self-amplifying RNA (replicon) in which the structural genes of VEE are replaced by a gene of interest, and the replicon RNA is packaged into VRP in cells by supplying the structural proteins in trans. In a preferred method, replicon RNA is packaged into VRP when cells are co-transfected with both replicon RNA and two separate helper RNAs, which together encode the full complement of VEE structural proteins. By using this approach, only the replicon RNA is packaged into VRP, as the helper RNAs lack the cis-acting packaging sequence required for encapsidation. Thus, VRP can infect target cells in culture or in vivo, and can express the gene of interest to high level from the subgenomic RNA. However, they are propagation defective in that they lack the critical portion of the VEE genome (i.e. the VEE structural protein genes) necessary to produce virus particles which could spread to other cells.

### SUMMARY OF THE INVENTION

The inventive subject matter of the present application relates to an immunogenic composition and method of immunizing a subject against malarial disease. The method consists of administering to the subject a priming immunization preparation containing one or more alphavirus replicons that express a gene or genes encoding a malarial antigen and then administering to the subject a boosting immunization preparation containing the malarial antigen, wherein the priming and boosting preparations express a full-length malarial antigen, an immunogenic fragment thereof, an epitope derived from the malarial antigen, or a combination thereof. The boosting immunization preparation can contain one or more of the following: a recombinant non-alphavirus viral expression system encoding the malarial antigen; a preparation of the malarial antigen produced by recombinant DNA technology; a synthetic preparation of the malarial antigen; a malarial organism or extract thereof; and a polynucleotide vector expressing the malarial antigen.

The inventive subject matter also relates to a method of enhancing and broadening immunogenicity and protective immunity against malaria, in mammals, that involves administering to a subject mammal a priming immunization preparation containing an alphavirus replicon or a combination of alphavirus replicons expressing a gene or genes encoding a malarial antigen and subsequently administering to the mammal a boosting immunization preparation that contains the malarial antigen, wherein the priming and boosting preparations express a full-length malarial antigen, an immunogenic fragment thereof, an epitope derived from the malarial antigen, or a combination thereof. The boosting immunization preparation can include one or more of the following: a recombinant non-alphavirus viral expression system encoding the malarial antigen; a preparation of the malarial antigen produced by recombinant DNA technology; a synthetic preparation of the malarial antigen; a malarial organism or extract thereof; and a polynucleotide vector expressing the malarial antigen.

The inventive subject matter of the present application further relates to an immunogenic composition and method of inducing an immune response that activates robust cellular and/or humoral responses.

The inventive subject matter also involves an immunogenic composition and method of inducing an immunogenic response against subdominant and immunodominant epitopes.

### BRIEF DESCRIPTION OF THE DRAWINGS

FIG. 1 demonstrates parasite-specific antibody responses to PyHEP17 induced by various routes of administration of PyHEP17 VEE-replicon particles (VRP) in homologous VRP vaccination strategies, as measured by indirect fluorescent antibody test (IFAT) against P. yoelii parasitized erythrocytes.

FIG. 2 demonstrates antigen-specific antibody responses to PyHEP17 or PyCSP induced by various routes of administration of PyHEP17 or PyCSP VRP in homologous VRP vaccination strategies, as measured by ELISA.

FIG. 3 demonstrates antigen-specific cell mediated immune responses to PyCSP induced by various routes of administration of PyCSP VRP in homologous VRP or heterologous VRP prime SQ vaccination strategies, as measured by intracellular cytokine staining or ELIspot.

FIG. 4 demonstrates parasite-specific antibody responses to PyHEP17 induced by various doses and numbers of immunization with PyHEP17 VRP in homologous VRP vaccination strategies, as measured by indirect fluorescent antibody test (IFAT) against P. yoelii parasitized erythrocytes.

FIG. 5a demonstrates parasite-specific antibody responses to PyCSP induced by PyCSP VRP using various routes of homologous VRP immunization or by heterologous VRP prime SQ vaccination strategies, as measured by ELISA.

FIG. 5b demonstrates parasite-specific antibody responses to PyHEP17 induced by PyHEP17 VRP using various routes of homologous VRP immunization or by heterologous VRP prime SQ vaccination strategies, as measured by ELISA.

FIG. 6 demonstrates the expression of cell phenotypic and activation markers induced by PyCSP VRP in homologous or heterologous VRP prime IM vaccination strategies, as measured by multiparameter flow cytometry.

FIG. 7 demonstrates antigen-specific cytokine responses to PyCSP induced by PyCSP VRP using various routes of homologous immunization or by heterologous VRP prime SQ vaccination strategies, as measured by intracellular cytokine staining or ELIspot.

FIG. 8 demonstrates the frequency and magnitude of antigen-specific cytokine responses to PyCSP induced by PyCSP VRP in homologous IM or heterologous VRP prime IM vaccination strategies, as measured by intracellular cytokine staining.

FIG. 9 demonstrates antigen-specific cytokine responses to PyCSP induced by PyCSP VRP in homologous IM or heterologous VRP prime IM vaccination strategies, as measured by ELIspot.

FIG. 10 demonstrates antigen-specific antibody responses to PyHEP17 induced by various doses of PyHEP17 VRP in homologous SQ or heterologous VRP prime SQ vaccination strategies, as measured by ELISA.

FIG. 11 demonstrates antigen-specific antibody responses to PyCSP induced by various doses by PyCSP VRP in homologous SQ or heterologous VRP prime SQ vaccination strategies, as measured by ELISA.

FIG. 12 demonstrates antigen-specific and parasite-specific antibody responses to PyCSP induced by various doses by PyCSP VRP in homologous IM or heterologous VRP prime IM vaccination strategies, as measured by ELISA against PyCSP antigen or indirect fluorescent antibody test (IFAT) against P. yoelii parasitized erythrocytes.

FIG. 13 demonstrates the capacity of homologous IM or heterologous VRP prime IM vaccination strategies with PyCSP VRP to protect against P. yoelii parasite challenge.

FIG. 14 demonstrates the correlation between CD8+ T cell IFNg responses induced by homologous or heterologous VRP prime IM vaccination strategies and protection against P. yoelii parasite challenge.

### DESCRIPTION OF THE PREFERRED EMBODIMENTS

Definitions

In the context of the present application: nm means nanometer; ml means milliliter; VEE means Venezuelan Equine Encephalitis virus; HA means hemaglutinin gene; GFP means green fluorescent protein gene; IFN means gamma-interferon; FACS means fluor-escence activated cell sorter; and FBS means Fetal Bovine Serum. The expression “E2 amino acid number” indicates designated amino acid at the designated residue of the E2 gene, and is also used to refer to amino acids at specific residues in the E1 gene.

As used herein, the term “alphavirus” has its conventional meaning in the art, and includes the various species such as VEE, Semliki Forest Virus (SFV), Sindbis, Ross River Virus, Western Equine Encephalitis Virus, Eastern Equine Encephalitis Virus, Chikungunya, S. A. AR86 (now referred to as “AR86”, to avoid confusion with the SARS coronavirus), Everglades virus, Mucambo, Barmah Forest Virus, Middelburg Virus, Pixuna Virus, O'nyong-nyong Virus, Getah Virus, Sagiyama Virus, Bebaru Virus, Mayaro Virus, Una Virus, Aura Virus, Whataroa Virus, Banbanki Virus, Kyzylagach Virus, Highlands J Virus, Fort Morgan Virus, Ndumu Virus, and Buggy Creek Virus. The preferred alphaviruses used in the constructs and methods of the claimed invention are VEE, AR86, Sindbis (e.g. TR339, see U.S. Pat. No. 6,008,035), and SFV.

The terms “5′ alphavirus replication recognition sequence” and “3′ alphavirus replication recognition sequence” refer to the sequences found in alphaviruses, or sequences derived therefrom, that are recognized by the nonstructural alphavirus replicase proteins and lead to replication of viral RNA. These are sometimes referred to as the 5′ and 3′ ends, or alphavirus 5′ and 3′ sequences. In the replicon constructs of the instant invention, the use of these 5′ and 3′ ends will result in replication of the RNA sequence encoded between the two ends. The 3′ alphavirus replication recognition sequence as found in the alphavirus is typically approximately 300 nucleotides in length, which contains a more well defined, minimal 3′ replication recognition sequence. The minimal 3′ replication recognition sequence, conserved among alphaviruses, is a 19 nucleotide sequence (Hill et al., Journal of Virology, 2693-2704, 1997). These sequences can be modified by standard molecular biological techniques to further minimize the potential for recombination or to introduce cloning sites, with the proviso that they must still be recognized by the alphavirus replication machinery.

The terms “alphavirus RNA replicon”, “alphavirus DNA replicon”, or collectively “alphavirus replicon” or “alphavirus vector replicon”, refer to a nucleic acid molecule expressing alphavirus nonstructural protein genes such that it can direct its own replication (amplification) and comprising, at a minimum, 5′ and 3′ alphavirus replication recognition sequences, coding sequences for alphavirus nonstructural proteins, a heterologous gene encoding an antigen and the means for expressing the antigen, and a polyadenylation tract. In the case of the alphavirus DNA replicon, the nucleic acid molecule also contains a 5′ promoter which can initiate transcription from the DNA in vivo (that is, within the subject to which the DNA replicon is administered).

Specific embodiments of the alphavirus replicons utilized in the claimed invention may contain one or more attenuating mutations, an attenuating mutation being a nucleotide deletion, addition, or substitution of one or more nucleotide(s), or a mutation that comprises rearrangement or chimeric construction which results in a loss of virulence in a live virus containing the mutation as compared to the appropriate wild-type alphavirus. Examples of locations for suitable attenuating mutations in the alphavirus VEE include the following: nucleotide 3, E2-76, preferably lysine, arginine, or histidine; E2-120, preferably lysine; E2-209, preferably lysine, arginine or histidine; E1-81, preferably isoleucine, E1-253 preferably serine; E1-272, preferably threonine or serine; and E3-56 to 59, preferably a deletion of all four amino acids. Mutations may also be introduced into the alphavirus genome to improve its functionality as a vaccine vector, e.g. a mutation in the region of E2 158-162, particularly E2-160 in the Sindbis strain to enhance its targeting to dendritic cells (See, for example, International PCT Publication No. WO 01/81609 Polo et al., published Nov. 1, 2001).

The terms “alphavirus structural protein/protein(s)” refers to one or a combination of the structural proteins encoded by alphaviruses. These are produced by the virus as a polyprotein and are represented generally in the literature as C-E3-E2-6k-E1. E3 and 6k serve as membrane translocation/transport signals for the two glycoproteins, E2 and E1. Thus, use of the term E1 herein can refer to E1, E3-E1, 6k-E1, or E3-6k-E1, and use of the term E2 herein can refer to E2, E3-E2, 6k-E2, or E3-6k-E2.

“Alphavirus replicon particle”, “recombinant alphavirus particles” or “alphavirus vector particle”, used interchangeably herein, mean a virion-like structural complex incorporating an alphavirus RNA replicon that expresses one or more heterologous RNA sequences. Typically, the virion-like structural complex includes one or more alphavirus structural proteins embedded in a lipid envelope enclosing a nucleocapsid that in turn encloses the RNA. The lipid envelope is typically derived from the plasma membrane of the cell in which the particles are produced. Preferably, the alphavirus replicon RNA is surrounded by a nucleocapsid structure comprised of the alphavirus capsid protein, and the alphavirus glycoproteins are embedded in the cell-derived lipid envelope. The alphavirus replicon particles are infectious but propagation-defective, i.e. the replicon RNA contained within the particle can replicate within the host cell that the particle infects, but it cannot direct the synthesis of additional replicon particles that could infect new host cells.

As used herein, the term “antigen” means an immunogenic peptide or protein which induces an immune response to a malarial pathogen capable of infecting a mammal. Antigens suitable for use in the present invention include, but are not limited to, the following Plasmodium genes: PfCSP, PfEXPI, PfSSP2, PfLSA-1, PfLSA-3, PfMSP-1, PfAMA-1, PfEBA-175, PfMSP-3, PfMSP-4, PfMSP-5, PfRAP-1, PfRAP-2, or other novel antigens defined by the Plasmodium falciparum genomic DNA sequence (See: Gardner, M. J. et al. (2002) Nature 419, 498-511), or their P. vivax, P. ovale, or P. malariae othologues. The entire sequence of the Plasmodium falciparum parasite is known (See Gardner, M. J. et al., supra), and any of the proteins encoding by this genome could theoretically function as antigens in this invention. The term “antigen” is further intended to encompass peptide or protein analogs of known or wild-type antigens such as those described above, which analogs may be more soluble or more stable than wild type antigen, and which may also contain mutations or modifications rendering the antigen more immunologically active. Further peptides or proteins that have sequences homologous with a desired antigen's amino acid sequence, where the homologous antigen induces an immune response to the respective pathogen, are also useful.

“Orthologues” and “Homologous Sequences” as used herein, refers to the subunit sequence similarity between two polymeric molecules, e.g., between two nucleic acid molecules, e.g., two DNA molecules or two RNA molecules, or between two polypeptide molecules. When a subunit position in both of the two molecules is occupied by the same monomeric subunit, e.g., if a position in each of two DNA molecules is occupied by adenine, then they are homologous at that position. The homology between two sequences is a direct function of the number of matching or homologous positions, e.g., if half (e.g. five positions in a polymer ten subunits in length) of the positions in two compound sequences are homologous then the two sequences are 50% homologous, if 90% of the positions, e.g., 9 of 10, are matched or homologous, the two sequences share 90% homology. By way of example, the DNA sequences 3′ATTGCC5′ and 3′TATGCG5′ share 50% homology. By the term “substantially homologous” as used herein, is meant DNA or RNA which is about 50% homologous, more preferably about 70% homologous, even more preferably about 80% homologous and most preferably about 90% homologous to the desired nucleic acid. Genes that are homologous to the desired antigen-encoding sequence should be construed to be included in the instant invention provided they encode a protein or polypeptide having a biological activity substantially similar to that of the desired antigen.

Analogs of the antigens described herein can differ from naturally occurring proteins or peptides by conservative amino acid sequence differences or through modifications that do not affect sequence, or by both. For example, conservative amino acid changes may be made, which although they alter the primary sequence of the protein or peptide, do not normally alter its function. Modifications (which do not normally alter primary sequence) include in vivo, or in vitro chemical derivatization of polypeptides, e.g., acetylation, or carboxylation. Also included as antigens are proteins modified by glycosylation, e.g., those made by modifying the glycosylation patterns of a polypeptide during its synthesis and processing or in further processing steps; e.g., by exposing the polypeptide to enzymes which affect glycosylation, e.g., mammalian glycosylating or deglycosylating enzymes. Also included as antigens according to this invention are sequences which have phosphorylated amino acid residues, e.g., phosphotyrosine, phosphoserine, or phosphothreonine. Also included as antigens are polypeptides that have been modified using ordinary molecular biological techniques so as to improve their resistance to proteolytic degradation or to optimize solubility properties. Analogs of such polypeptides include those containing residues other than naturally occurring L-amino acids, e.g., D-amino acids or non-naturally occurring synthetic amino acids. The antigens of the invention are not limited to products of any of the specific exemplary processes listed herein.

The term “immune response” or “immunization” refer to the development in a subject of a humoral and/or cellular immunological response to an antigen that has been administered to the subject by the methods of this invention. “Humoral” immune responses refer to the production of antibodies, and a “cellular” immune response refers to the activation of T-lymphocytes, particularly cytolytic T-cells (“CTLs”) and helper T-cells. Specific T-cells involved in the cellular immune response include CD4+ and CD8+T-cells. The term “homologous immunization” as used herein, refers to method whereby the priming immunization and the boosting immunization are with the same different vaccine technology or vaccine delivery system. The term “heterologous immunization” as used herein, refers to method whereby the priming immunization and the boosting immunization are with a different vaccine technology or vaccine delivery system.

To stimulate the humoral arm of the immune system, i.e. the production of antigen-specific antibodies, an “immunogenic fragment” will generally include at least about 5-10 contiguous amino acid residues of the full-length molecule, preferably at least about 15-25 contiguous amino acid residues of the full-length molecule, and most preferably at least about 20-50 or more contiguous amino acid residues of the full-length molecule, that define an epitope, or any integer between 5 amino acids and the full-length sequence, provided that the fragment in question retains immunogenic activity, as measured by an assay, such as the ones described herein.

Regions of a given polypeptide that include an “epitope” can be identified using any number of epitope mapping techniques, well known in the art. (See, e.g., Epitope Mapping Protocols in Methods in Molecular Biology, Vol. 66, Glenn E. Morris, Ed., 1996, Humana Press, Totowa, N.J.) For example, linear epitopes can be determined by e.g., concurrently synthesizing large numbers of peptides on solid supports, the peptides corresponding to portions of the protein molecule, and reacting the peptides with antibodies while the peptides are still attached to the supports. Such techniques are known in the art and described in, e.g., U.S. Pat. No. 4,708,871; Geysen et al. (1984) Proc. Natl. Acad. Sci. USA 81:3998-4002; Geysen et al. (1986) Molec. Immunol. 23:709-715, all incorporated herein by reference in their entireties. Similarly, conformational epitopes are readily identified by determining spatial conformation of amino acids such as by, e.g., x-ray crystallography and 2-dimensional nuclear magnetic resonance. See, e.g., Epitope Mapping Protocols, supra. Antigenic regions of proteins can also be identified using standard antigenicity and hydropathy plots, such as those calculated using, e.g., the Omiga version 1.0 software program available from the Oxford Molecular Group. This computer program employs the Hopp/Woods method (Hopp et al., Proc. Natl. Acad. Sci USA (1981) 78:3824-3828) for determining antigenicity profiles and the Kyte-Doolittle technique (Kyte et al., J. Mol. Biol. (1982) 157:105-132) for hydropathy plots.

Generally, T-cell epitopes which are involved in stimulating the cellular arm of the subject's immune system, are short peptides of 8-25 amino acids, and these are not typically predicted by the above-described methods for identifying humoral epitopes. A common way to identify T-cell epitopes is to use overlapping synthetic peptides and analyze pools of these peptides, or the individual ones, that are recognized by T cells from animals that are immune to the antigen of interest, using an enzyme-linked immunospot assay (ELISPOT). These overlapping peptides can also be used in other assays such as the stimulation of cytokine release or secretion, or by the ability to interact with major histocompatibility (MHC) tetramers. Such immunogenic fragments can also be identified based on their ability to stimulate lymphocyte proliferation in response to stimulation by various fragments from the antigen of interest.

The term “epitope” as used herein refers to a sequence of at least about 3 to 5, preferably about 5 to 10 or 15, and not more than about 1,000 amino acids (or any integer therebetween), which define a sequence that by itself or as part of a larger sequence, binds to an antibody generated in response to such sequence or stimulates a cellular immune response. There is no critical upper limit to the length of the immunogenic fragment, which may comprise nearly the full-length of the protein sequence, or even a fusion protein comprising two or more epitopes from a single or multiple malarial parasite proteins. An epitope for use in the subject invention is not limited to a polypeptide having the exact sequence of the portion of the parent protein from which it is derived. Indeed, there are many known species of Plasmodium and the parasite retains the ability to continue to adapt, and there are several variable domains in the parasite that exhibit relatively high degrees of variability between species. Thus the term “epitope” encompasses sequences identical to the native sequence, as well as modifications to the native sequence, such as deletions, additions and substitutions (generally conservative in nature).

Immunization Methods

One component of the methods and compositions of the present invention is the use of a “priming” immunization, comprising the initial administration of one or more antigens to an animal, especially a human patient, in preparation for subsequent administration(s) of the same antigen. Specifically, the term “priming”, or alternatively “initiating” or “activating” an immune response or “enhancing” and “potentiating”, as used herein, defines a first immunization using an antigen which induces an immune response to the desired antigen and recalls a higher level of immune response to the desired antigen upon subsequent re-immunization with the same antigen when administered in the context of the same or a different vaccine delivery system. Specifically as used in this application, a “priming immunization” refers to the administration of a composition comprising an alphavirus replicon capable of expressing a malarial antigen. As used herein, a “priming immunogenic composition” refers to a composition of alphaviral replicons used to prime the immune system of the animal.

Another component of the methods and compositions of the present invention is the use of a “boosting immunization”, or a “boost”, which means the administration of a composition delivering the same malarial antigen as encoded in the priming immunization. A boost is sometimes referred to as an anamnestic response, i.e. an immune response in a previously sensitized animal. Multiple boosts can be administered, utilizing the same or differing amounts for each boost. Encompassed in the instant invention is the requirement that at least one of the ‘boosting’ immunizations utilize a composition that does not contain an alphavirus replicon. This ‘boosting’ immunization can be referred to as a ‘heterologous boost’ because it is different from the priming immunization.

In specific embodiments of the invention, the boosting immunization can be a recombinant non-alphavirus virus expression system, a recombinant malarial antigen preparation, a preparation of whole malarial parasite(s) or extracts thereof, or a polynucleotide vector, wherein said boosting preparation expressed either full-length malarial antigen, an immunogenic fragment thereof, or an epitope derived from the malarial antigen. As an example of one embodiment of this invention, the priming immunization is a VEE replicon particle expressing the malarial antigen, and the boost is a pox virus(vaccinia) vector expressing the same malarial antigen.

The widespread deployment of subunit vaccines may depend upon the use of immunization regimes in which the vaccine is administered together with recombinant alphaviruses (replicons of Venezulan Encephalitis Virus, Semliki Forest virus or Sindbis virus, for instance, and others like them) recombinant poxviruses (vaccinia, canarypox, cowpox, fowlpox, monkeypox, for instance, and others like them); recombinant adenoviruses or adeno-associated virus; recombinant proteins; synthetic peptides; plasmid DNA; or live, attenuated or killed organisms, or extracts thereof. In the instant invention, it has been determined that immunizing with a priming immunization containing an alphavirus replicon vaccine or combination of alphavirus replicon vaccines and subsequently immunizing with a boosting immunization preparation that contains one or more recombinant poxviruses; recombinant adenoviruses; recombinant proteins; synthetic peptides; plasmid DNA, or live, attenuated or killed organisms, or extracts thereof, provides a method of immunization that induces a broad immunogenic response to the encoded antigen and protective immunity against parasite challenge. As a result of this method, the amount (dose) of vaccine needed to achieve protective immunity may be reduced, the duration of vaccine-induced protective immunity may be increased, and the vaccine coverage in a vaccinated population may be broadened.

The Alphavirus genus includes a variety of viruses, all of which are members of the Togaviridae family. The alphaviruses include Eastern Equine Encephalitis virus (EEE), Venezuelan Equine Encephalitis virus (VEE), Everglades virus, Mucambo virus, Pixuna virus, Western Equine Encephalitis virus (WEE), Sindbis virus, Semliki Forest virus, Middleburg virus, Chikungunya virus, O'nyong-nyong virus, Ross River virus, Barmah Forest virus, Getah virus, Sagiyama virus, Bebaru virus, Mayaro virus, Una virus, Aura virus, Whataroa virus, Babanki virus, Kyzylagach virus, Highlands J virus, Fort Morgan virus, Ndumu virus, and Buggy Creek virus. The viral genome is a single-stranded, messenger-sense RNA, modified at the 5′-end with a methylated cap and at the 3′-end with a variable-length poly (A) tract. Structural subunits containing a single viral protein, capsid, associate with the RNA genome in an icosahedral nucleocapsid. In the virion, the capsid is surrounded by a lipid envelope covered with a regular array of transmembrane protein spikes, each of which consists of a heterodimeric complex of two glycoproteins, E1 and E2. See Pedersen et al., J. Virol 14:40 (1974). The Sindbis and Semliki Forest viruses are considered the prototypical alphaviruses and have been studied extensively. See Schelsinger, The Togaviridae and Flaviviridae, Plenum Publishing Corp., New York (1986). The VEE virus has also been studied extensively (See, e.g., U.S. Pat. No. 5,185,440).

The complete genomic sequences, as well as the sequences of the various structural and non-structural proteins are known in the art for numerous alphaviruses and include: Sindbis virus genomic sequence (GenBank Accession Nos. J02363, NCBI Accession No. NC_{—}001547), S.A.AR86 genomic sequence (GenBank Accession No. U38305), VEE genomic sequence (GenBank Accession No. L04653, NCBI Accession No. NC_{—}001449), Girdwood S.A genomic sequence (GenBank Accession No. U38304), Semliki Forest virus genomic sequence (GenBank Accession No. X04129, NCBI Accession No. NC_{—}003215), and the TR339 genomic sequence (See Klimstra et al., (1988) J. Virol. 72:7357; and McKnight et al., (1996) J. Virol. 70:1981; the disclosures of which are incorporated herein by reference in their entireties).

The studies of these viruses have led to the development of techniques for vaccinating against diseases through the use of alphavirus vectors for the introduction of genes expressing antigens derived from the organism(s) causing disease. The alphavirus replicon system, as described in U.S. Pat. No. 6,190,666 to Garoff et al., U.S. Pat. Nos. 5,792,462 and 6,156,558 to Johnston et al., U.S. Pat. Nos. 5,814,482, 5,843,723, 5,789,245, 6,015,694, 6,105,686 and 6,376,236 to Dubensky et al; U.S. Published Application No. 2002/0015945 A1 (Polo et al.), U.S. Published Application No. 2001/0016199 (Johnston et al.), Frolov et al. (1996) Proc. Natl. Acad. Sci. USA 93:11371-11377 and Pushko et al. (1997) Virology 239:389-401, is particularly well-suited as a vector for delivering disease organism antigens. The alphavirus replicon vector system can be delivered in the form of DNA replicons, which are “launched” within the host cell to express the replicon RNA which then expresses the antigen. Similarly, naked RNA replicons can be introduced directly into the host cell, e.g. by transfection of patient's cells in an ex vivo manner. Alternatively, the replicon can be delivered to the subject as an alphavirus replicon particle. In all of these embodiments, the replicon RNA contains sequences required for replication and packaging of the RNA into a virus-like particle. It contains a nonstructural proteins open reading frame (ORF), which provides viral protein required for genome replication and transcription of subgenomic RNA, but lacks the structural protein genes necessary for formation of viral particle. The replicon is engineered so that the subgenomic RNA contains ORF(s) coding for a gene of interest, in this invention one or more genes encoding malarial antigens. To produce the alphavirus replicon particles, one or more helper nucleic acids provide the alphavirus capsid and glycoprotein genes. The replicon RNA vector and the one or more helper nucleic acids are introduced into an alphavirus-permissive cell, the replicon RNA is packaged into virus-like particles, which are harvested and purified from these cells to produce an immunogenic preparation, i.e. a vaccine composition.

Immunogenic Composition Dosage and Routes of Administration

Pharmaceutical formulations, such as vaccines or other immunogenic compositions, of the present invention comprise an immunogenic amount of the priming immunization preparation, the boosting immunization preparation, or both, in combination with a pharmaceutically acceptable carrier. An “immunogenic amount” is an amount of the preparation(s) which is sufficient to evoke an immune response in the subject to which the pharmaceutical formulation is administered. The immunogenic preparations are administered in a manner compatible with the dosage formulation, and in such amount as will be prophylactically and/or therapeutically effective. In the case of the priming immunization comprising alphavirus replicon particles (ARPs), e.g. VEE replicon particles (VRP), an amount of from about 10⁴ to about 10⁹ infectious units or VRPs per dose is believed suitable, depending upon the subject to be treated, the route by which the VRPs are administered, the immunogenicity of the expression product, the types of effector immune responses desired, and the degree of protection desired. Precise amounts of the active ingredient required to be administered may depend on the judgment of the physician, veterinarian or other health practitioner and may be peculiar to each individual, but such a determination is within the skill of such a practitioner.

Exemplary pharmaceutically acceptable carriers include, but are not limited to, sterile pyrogen-free water and sterile pyrogen-free physiological saline solution. Immunogenic compositions comprising the VRPs may be formulated by any of the means known in the art. Such compositions, especially vaccines, are typically prepared as injectables, either as liquid solutions or suspensions. Solid forms suitable for solution in, or suspension in, liquid prior to injection may also be prepared.

The active immunogenic ingredients (e.g.VRPs) are often mixed with excipients or carriers which are pharmaceutically acceptable and compatible with the active ingredient. Suitable excipients include but are not limited to sterile water, saline, dextrose, glycerol, ethanol, or the like and combinations thereof, as well as stabilizers, e.g. Human Serum Albumin (HSA) or other suitable proteins and reducing sugars.

In addition, if desired, the vaccines may contain minor amounts of auxiliary substances such as wetting or emulsifying agents, pH buffering agents, and/or adjuvants which enhance the effectiveness of the vaccine. Examples of adjuvants which may be effective include but are not limited to: aluminum hydroxide; N-acetyl-muramyl-L-threonyl-D-isoglutamine (thr-MDP); N-acetyl-nor-muramyl-L-alanyl-D-isoglutamine (CGP 11637, referred to as nor-MDP); N-acetylmuramyl-L-alanyl-D-isoglutaminyl-L-alanine-2-(1′-2′-dipalmitoyl-sn-glycero-3hydroxyphosphoryloxy)-ethylamine (CGP 19835A, referred to as MTP-PE); MF59 (see International Publication No. WO90/14837); RIBI (Corixa, Seattle Wash.), which contains three components extracted from bacteria, monophosphoryl lipid A, trehalose dimycolate and cell wall skeleton (MPL+TDM+CWS) in a 2% squalene/Tween 80 emulsion; and saponins, e.g. Stimulon® (Cambridge Bioscience, Worcester, Mass.). The effectiveness of an adjuvant may be determined by measuring the amount of antibodies and/or T cell responses directed against the malarial antigen resulting from administration of the immunizing preprations of this invention which are also comprised of one or more adjuvants. Such additional formulations and modes of administration as are known in the art may also be used.

Subjects which may be administered the priming and boosting pharmaceutical formulations of this invention include human and animal (e.g., dog, cat, cattle, horse, donkey, mouse, hamster, monkeys, guinea pigs, birds, eggs) subjects.

As used herein, priming immunizations of this invention are given in a single dose. The boosting immunization can comprise one or more boosting doses, for example, 1 to 5 separate doses, administered at subsequent time intervals as required to induce, maintain, enhance and/or reinforce the immune response, e.g., weekly or at 1 to 4 months, and if needed, a subsequent dose(s) after several weeks, months or years. Each priming or boosting preparation may express either full-length malarial antigen, an immunogenic fragment thereof, an epitope derived from the malarial antigen, or a combination thereof.

All references cited in the present application are incorporated by reference herein to the extent that there is no inconsistency with the present disclosure.

In the P. yoelii rodent malaria model, PyCSP and PyHEP17 are target antigens of protective CD8+ and CD4+ T cell responses and protective antibody responses. Therefore, recombinant PyCSP and PyHEP17 alphavirus vaccines in mice provided an ideal experimental system with which to measure the enhancing effect of boosting with one or more recombinant alphavirus vaccines subsequent to a priming immunization with a heterologous vaccine. Some examples of different immunization regimes are depicted in the specific examples.

Having described the invention, the following examples are given to illustrate specific applications of the invention including the best mode now known to perform the invention. These specific examples are not intended to limit the scope of the invention described in this application.

### WORKING EXAMPLES

### Example 1

### Improved Immunogenicity in a Mouse Model of Malaria

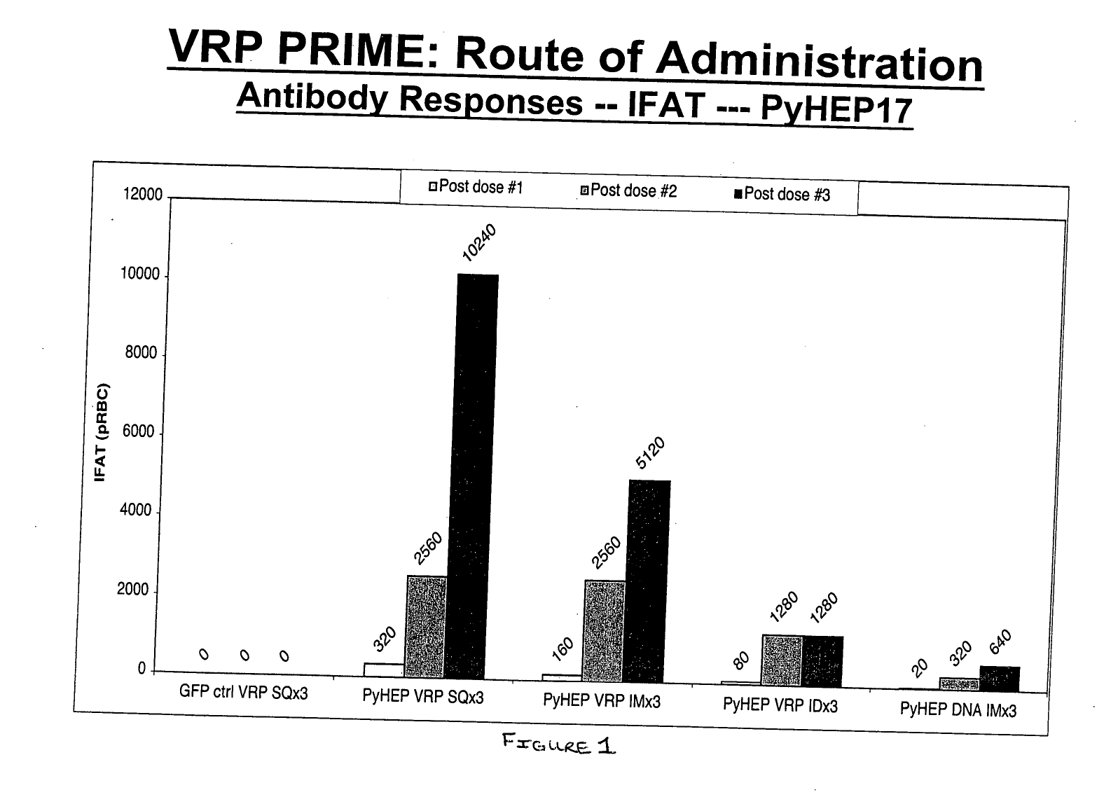

FIG. 1 demonstrates parasite-specific antibody responses to PyHEP17 induced by various routes of administration of PyHEP17 VRP in homologous VRP vaccination strategies, as measured by indirect fluorescent antibody test (IFAT) against P. yoelii parasitized erythrocytes.

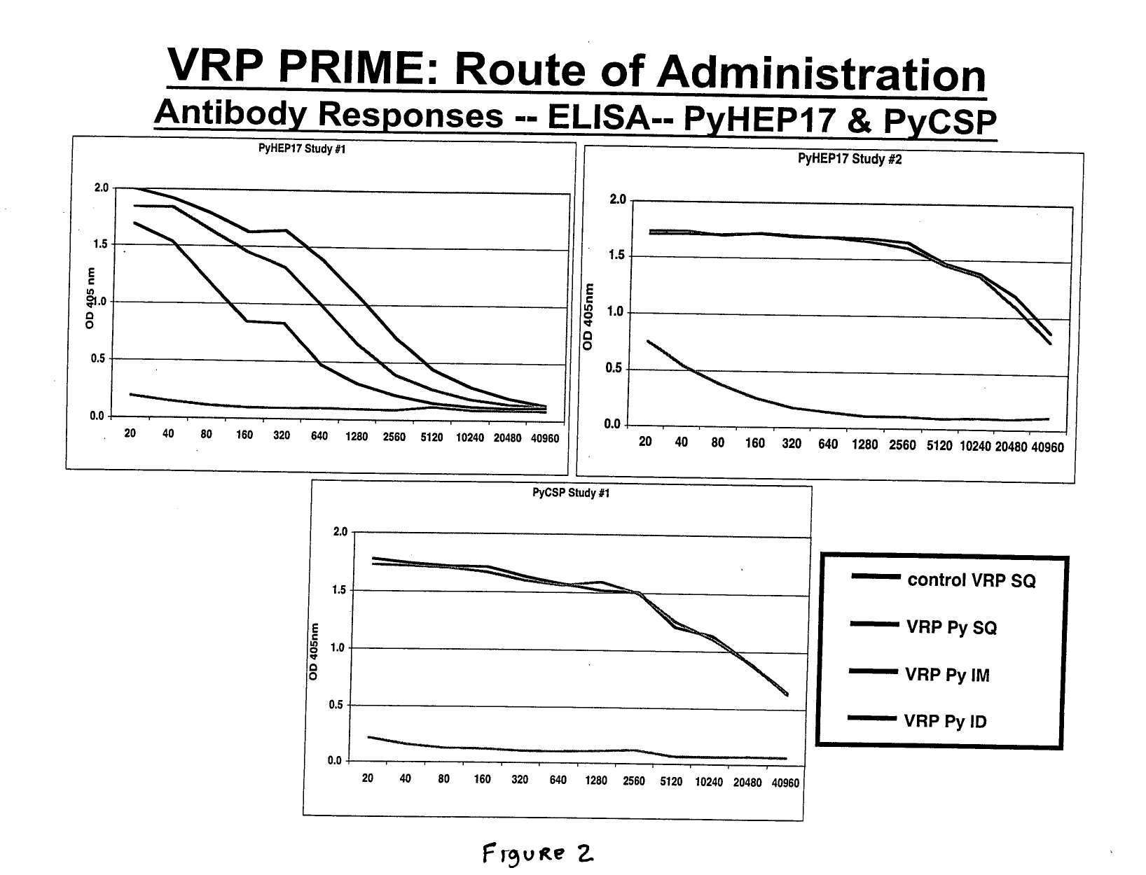

FIG. 2 demonstrates antigen-specific antibody responses to PyHEP17 or PyCSP induced by various routes of administration of PyHEP17 or PyCSP VRP in homologous VRP vaccination strategies, as measured by ELISA.

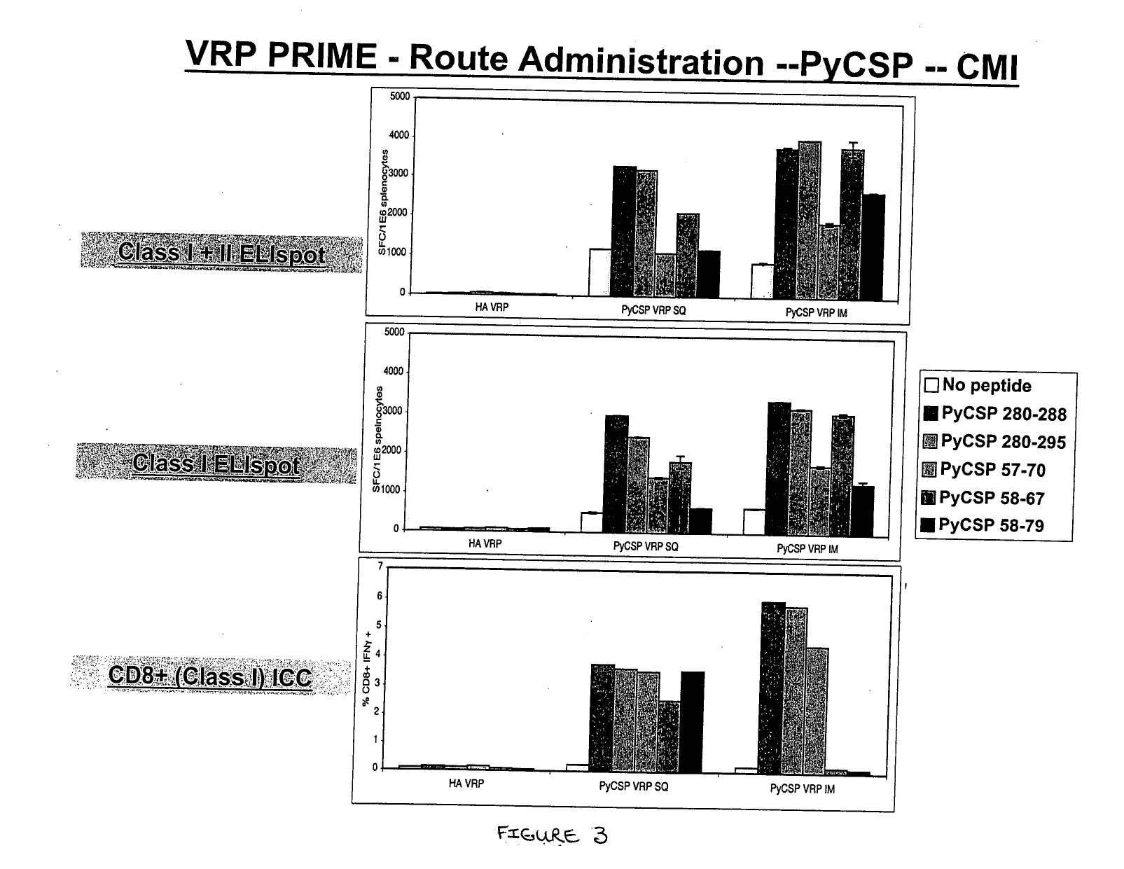

FIG. 3 demonstrates antigen-specific cell mediated immune responses to PyCSP induced by various routes of administration of PyCSP VRP in homologous VRP or heterologous VRP prime SQ vaccination strategies, as measured by intracellular cytokine staining or ELIspot.

Study:

Female 4- to 8-wk-old BALB/cByJ (H-2d) (cat# JR001026) mice were obtained from The Jackson Laboratory, Bar Harbor, Me. Groups of 6 mice were immunized with 5×10⁶ IFU of VEE viral replicon particles (VRP) encoding the malaria gene of interest (PyHEP17 or PyCSP) or an unrelated control gene (GFP) three times at 4 week intervals by subcutaneous (SC) (footpad, 50 μl×2 sites), intramuscular (IM) (tibialis anterior, 50 μl×2 sites) or intradermal (ID) (back, 25 μl×4 sites) routes of administration. For intramuscular injections, the vaccines were injected into each tibialis anterior muscle in a volume of 50 μl PBS, using a 0.3 ml insulin syringe fitted with a plastic collar cut from a micropipette tip, adjusted to limit the needle penetration to a distance of about 2 mm into the muscle. For intradermal injection, the back of the mice was shaved and 100 μl of vaccine was split and injected into four sites of the skin (25 μl per site) as shallow as possible in the epidermis to generate four blebs, using a 0.3 ml insulin syringe. For subcutaneous injection, 100 μl vaccine were injected into the footpad of the mouse, using a 0.3 ml insulin syringe. Sera were collected 3 weeks after each immunization, and assayed for parasite-specific antibody responses by indirect fluorescent antibody test (IFAT) against P. yoelii parasitized erythrocytes (FIG. 1) or antigen-specific antibodies by ELISA against PyHEP17 synthetic peptide or PyCSP recombinant protein (FIG. 2). Splenocytes were harvested at 3 weeks after the third immunization, and assayed for antigen specific cell mediated immune responses by IFNg ELIspot or intracellular cytokine staining flow cytometry assays (FIG. 3).

Methods:

Antibody ELISA assays: _Mice were bled from tail vein for serum approximately 2-3 wk after each immunization. Antibodies were measured by enzyme-linked immunosorbent assay (ELISA) against recombinant PyCSP protein PyHEP17 MR68 peptide or PyHEP17 recombinant protein, as previously described (Charoenvit et al., 1987; 1999). In addition, antibodies were assessed by the indirect fluorescent antibody test (IFA) against air-dried P. yoelii sporozoites for PyCSP or air-dried P. yoelii infected erythrocytes for PyHEP17, as previously described (Charoenvit et al., 1987). Briefly, for PyCSP, 50 μl of 0.1 μg/ml of recombinant protein in PBS was added into wells of Immunolon II ELISA plates (Dynatech Laboratory Inc., Chantilly, Va.) and incubated for 6 h at room temperature. Wells were washed 4 times with PBS containing 0.05% Tween 20 (washing buffer) and incubated overnight at 4° C. with 100 μl of 5% nonfat dry milk in PBS (blocking buffer). After washing 4 times with washing buffer, the wells were incubated for 2 hr with 50 μl of 2-fold serial dilutions of mouse serum or a 1:20 dilution of supernatant mAb NYSI (for PytCSP) diluted in PBS containing 3% nonfat dry milk (diluting buffer). The wells were again be washed 4 times, incubated for 1 hr with peroxidase-labeled goat anti-mouse IgG (Kirkegaad & Perry, Gaithersburg, Md.) diluted 1:2000 in diluting buffer, then again washed 4 times. The wells were incubated for 20 min with 100 μl of a solution containing ABTS substrate [2,2′-azino-di-(3 ethylbenzthiazoline sulfonate] (Kirkegaard & Perry, Gaithersburg, Md.) and H₂O₂. Color reaction was measured in a micro-ELISA automated reader at OD 410 nm. All reaction steps except blocking were performed at room temperature. Data are presented as the OD reading for each reciprocal serum dilution.

Antibody IFAT assays: Immunofluorescence assays were carried out using air dried P. yoelii sporozoites (for PyCSP) or parasitized erythrocytes (for PyHEP17) as previously described (Charoenvit et al., 1987; 1999). Results are presented as the reciprocal of the last serum dilution at which fluorescence was scored as positive.

Interferon γELISPOT: Multiscreen MAHAS 4510 plates (Millipore, Bedford, Mass.) were coated with 60 μl/well of sterile carbonate/bicarbonate buffer containing 10 μg/ml of anti-murine IFN-γ (R4, Pharmingen, San Diego, Calif.) and incubated overnight at room temperature. Plates were washed twice with 200 μl/well RPMI medium and twice with cRPMI medium containing Penicillin/Streptomycin, L-Glutamine and 10% FBS, and incubated with 200 μl/well of cRPMI medium in 5% CO₂ at 37° C. for at least 3 hr. After blocking, the plates were washed once more with cRPMI before the addition of target and effector cells. A20.2J (e.g., ATCC clone HB-98) or P815 (ATCC TIB 64) target cells were washed once with cRPMI, incubated at 5×10⁶ cells/ml with or without synthetic peptides representing immunodominant or subdominant CD8+ or CD4+ T cell epitopes derived from PyCSP or PyHEP17 (10 μg/ml) for 1 hr at 37° C. in 5% CO₂, and irradiated in a 1³⁷CS gamma irradiator (A20.2J at 16,000 rads and P815 at 10,000 rads). Next, target cells were washed 3 times with cRPMI, diluted to 1.0×10 cells/ml (P815) or 1.5×10⁶ cells/ml (A20.2J) in cRPMI. To obtain splenocytes, immunized mice were sacrificed 2-7 wks after the final immunization (3-6 mice/group), their spleens removed to a sterile tissue screen and ground with glass pestle into a sterile petri dish using cRPMI. The spleen cell suspensions were washed 3 times, counted and diluted to 5×10⁶ cells/ml and 2.5×10⁶ cells/ml. Both spleens and target cells were plated in quadruplicates at 100 μl/well, and incubated in 5% CO₂ at 37° C. for 36 h. Plates were washed 3 times with PBS followed by 4 times with PBS-T (PBS 0.05% Tween20). Then, 100 μl/well of biotinylated anti-IFN-γ (XMG1.2, Pharmingen, San Diego, Calif.) at 2 μg/ml in PBS-T was added to each well and the plate was incubated overnight at 4° C. Plates were washed 6 times with PBS-T and then 100 μl/well peroxidase conjugated streptavidin (Kirkekaard & Perry, Gaithesburg, Md.) was added at 1:800 dilution in PBS-T. After 1 hr incubation at room temperature, plates were washed 6 times with PBS-T followed by 3 times with PBS alone, and developed with DAB reagent (Kirkekaard & Perry, Gaithesburg, Md.) according to manufacturer's instructions. After 15 min, the plates were rinsed extensively with dH₂O to stop the colorimetric substrate, dried and stored in the dark. Spots were counted using an automated KS Elispot reader (Carl Zeiss Vision, Germany).

Intracellular cytokine staining and FACS analysis: A20.2J cells were pulsed with or without synthetic peptides representing immunodominant or subdominant CD8+ or CD4⁺ T cell epitopes derived from PyCSP or PyHEP17 (10 μg/ml) for 1 hr at 37° C. in 5% CO₂, and irradiated in a ¹³⁷Cs gamma irradiator (A20.2J at 16,000 rads and P815 at 10,000 rads). Then, 100 μl/well of spleen cells (5×10⁶ cells/ml) and 100 μl/well A20.2J cells (1.5×10⁶ cells/ml) pulsed with or without PyCSP peptide were incubated in duplicates in U-bottom 96-well plates (Costar) in the presence of 1 μM Brefeldin A (GolgiPlug™, Pharmingen, San Diego, Calif.) in 5% CO₂ at 37° C. for 16 h. Plates were spun at 1,200 rpm for 5 min, the supernatant flicked, and the cell pellet resuspended by gentle vortexing. Cell surface markers were stained with 0.3-0.5 μl/well of anti-CD8-APC, anti-CD4-PERCP, or antibodies against CD62L, CD69, CD25, CD43, CD71, CD152 Abs (Pharmingen, San Diego, Calif.) in a final volume of 100 μl in FACS wash. Plates were incubated with a combination of three Abs on ice in the dark for 20 min. After the surface staining, cells were washed with FACS wash twice, gently resuspended, and incubated with 90 μl of Perm/Fix buffer (Pharmingen, San Diego, Calif.) for 20 min on ice in the dark. Next, cells were washed with 100 μl of Perm/Wash buffer and intracellular IFN-γ or TNF-α were stained with 0.5 μl/well of anti-IFN-γ-PE or anti-TNF-α-PE Abs (Pharmingen, San Diego, Calif.) in a final volume of 100 μl in Perm/Wash buffer. After 20 min incubation on ice in the dark, cells were washed twice with Perm/Wash, once with FACS wash, resuspended in 100 μl of FACS wash and stored at 4° C. prior to analysis. The frequency of cells secreting IFN-γ and TNF-α was determined by four-color fluorescent activated cell sorting using the FACSCalibur™ (Becton Dickinson Immunocytometry Systems, San Jose, Calif.).

Data presented in FIG. 1 demonstrate antibody responses induced by homologous immunization with PyHEP17 viral replicon particles (VRPs) by either subcutaneous (SQ), intramuscular (IM) or intradermal (ID) routes of administration, as measured in sera collected after one, two or three immunizations by an indirect fluorescent antibody test (IFAT) against P. yoelii parasitized erythrocytes. Also depicted are the response induced by homologous immunization with PyHEP17 plasmid DNA or control (GFP) VRPs. Data show results with pooled sera (n=6) collected after each immunization.

Data presented in FIG. 2 demonstrate antibody responses induced by homologous immunization with PyHEP17 or PyCSP viral replicon particles (VRPs) by either subcutaneous (SQ), intramuscular (IM), or intradermal (ID) routes of administration, as measured in sera collected after three immunizations by ELISA against a synthetic peptide representing the immunodominant PyHEP17 B cell epitope or against PyCSP recombinant protein as capture antigen. Also depicted is the response induced by homologous immunization with control (GFP) VRP. Data show results with pooled sera (n=6) collected after the third immunization.

Data presented in FIG. 3 demonstrate antigen-specific cytokine (IFNg) responses induced by homologous immunization with PyCSP viral replicon particles (VRPs) by either subcutaneous (SQ) or intramuscular (IM) routes of administration, using splenocytes collected after three immunizations as effector cells in ELIspot or intracellular cytokine flow cytometry (ICC, CD8+gated population) assays with MHC-matched target cells expressing either class I (P815 cells) or class I and class II (A20.2J cells) pulsed with synthetic peptides representing defined PyCSP CD8+ and/or CD4+ T cell epitopes or without peptide. PyCSP residues 280-288=dominant CD8+T cell epitope; PyCSP residues 280-295, overlapping dominant CD4+ and dominant CD8+T cell epitope; PyCSP residues 57-70=dominant CD4+ T cell epitope; PyCSP residues 58-67=subdominant CD8+T cell; and PyCSP residues 58-79=overlapping dominant CD4+ T cell epitope and subdominant CD8+ T cell epitope. Also depicted are the responses induced by homologous immunization with control (GFP) VRPs. Data show results with pooled splenocytes (n=6) collected after the third immunization. ELIspot data are plotted as spot forming cells (SFC) per million splenocytes. ICC data are presented as the frequency of CD8+T cells secreting IFNg.

Results represented in FIGS. 1, 2, and 3 show that VEE viral replicon particles expressing malaria antigens of interest administered SQ or IM or ID can induce antigen-specific and parasite specific antibody responses. SQ and IM routes of administration are markedly more effective than ID immunization, but all three routes are more effective at inducing antibodies than immunization with plasmid DNA. VRP expressing malaria antigens of interest administered SQ or IM can also induce as antigen-specific cell mediated immune responses against both immunodominant and subdominant CD8+ and CD4+ T cell epitopes. Overall, data demonstrate that VEE viral replicon particles expressing malaria antigens can effectively activate both humoral and cellular arms of the immune system, and induce a profile of broad epitope recognition. Significantly, VEE replicon particles demonstrate the capacity to effectively induce antibody responses against those antigens (e.g., PyHEP17) for which antibodies have been difficult to induce using other vaccine delivery platforms.

### Example 2

Immunogenicity of VEE Viral Replicon Particles in a Mouse Model of malaria—Number of Immunizations and Number of Doses

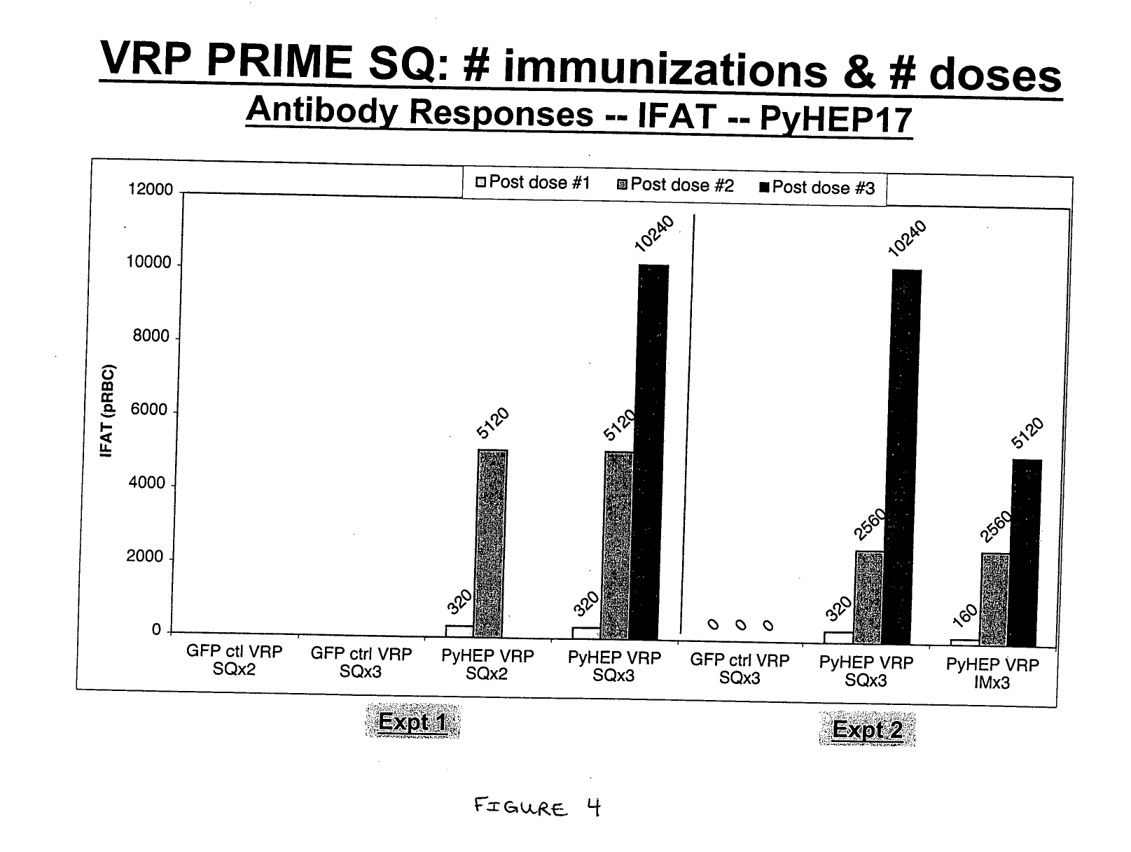

FIG. 4 demonstrates antigen-specific antibody responses to PyHEP17 induced by various doses and numbers of immunization with PyHEP17 VRP in homologous VRP vaccination strategies, as measured by indirect fluorescent antibody test (IFAT) against P. yoelii parasitized erythrocytes.

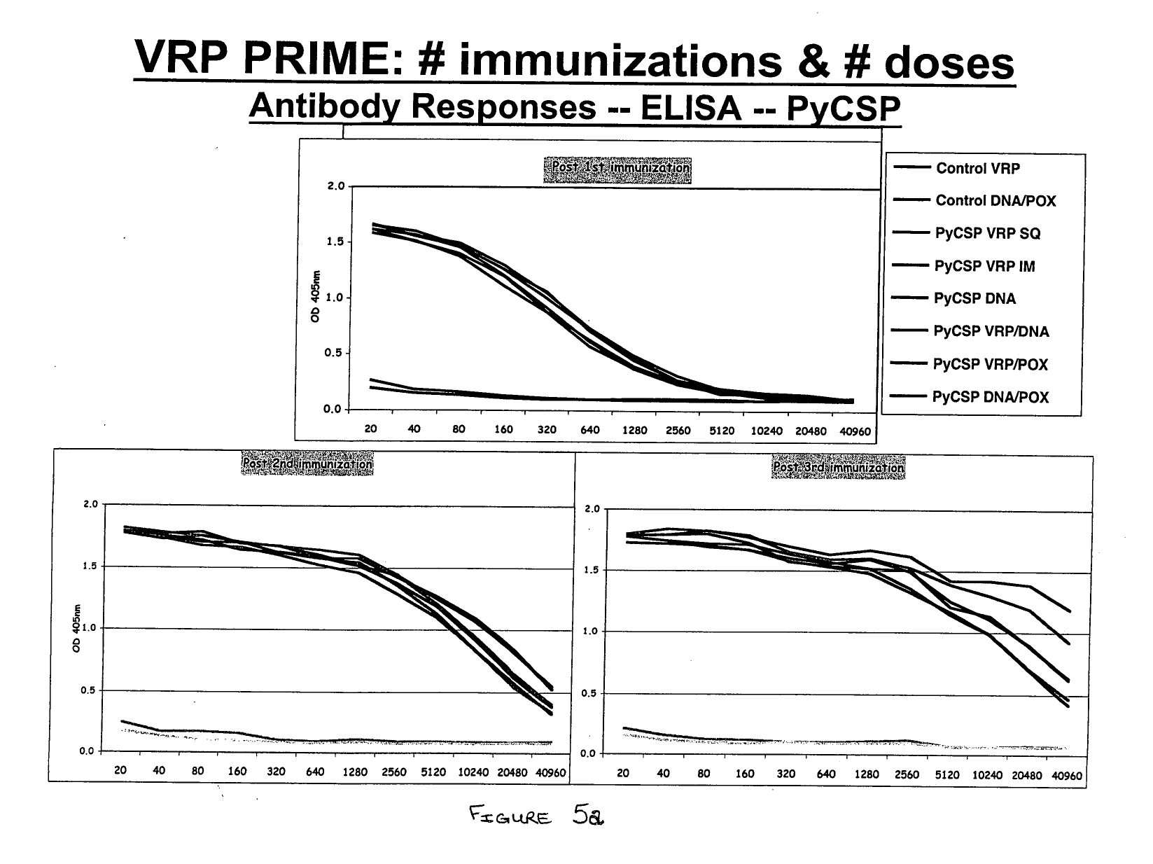

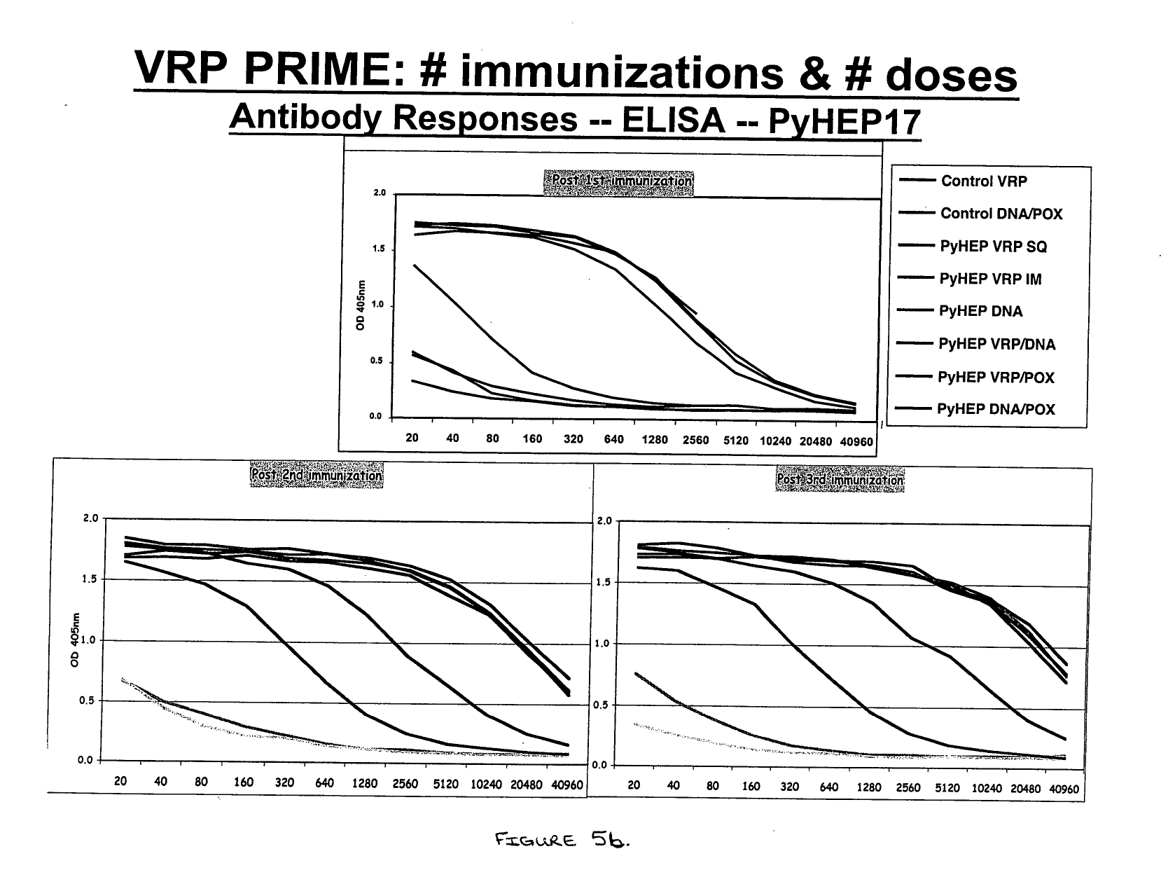

FIG. 5a demonstrates parasite-specific antibody responses to PyCSP induced by PyCSP VRP using various routes of homologous VRP immunization or by heterologous VRP prime SQ vaccination strategies, as measured by ELISA.

FIG. 5b demonstrates parasite-specific antibody responses to PyHEP17 induced by PyHEP17 VRP using various routes of homologous VRP immunization or by heterologous VRP prime SQ vaccination strategies, as measured by ELISA.

Study:

Female 4- to 8-wk-old BALB/cByJ (H-2d) (cat# JR001026) mice were obtained from The Jackson Laboratory (Bar Harbor, Me._. Groups of 6 mice were immunized with 5×10⁶ VEE viral replicon particles (VRPs) encoding the malaria gene of interest (PyHEP17 or PyCSP) or an unrelated control gene (GFP) two or three times at 4 week intervals by subcutaneous (SC) (footpad, 50 μl×2 sites) or intramuscular (IM) (tibialis anterior, 50 μl×2 sites) routes of administration. Additionally, some groups of mice were immunized two times at 4 week intervals by the subcutaneous (SC) (footpad, 50 μl×2 sites) route and then boosted IM with plasmid DNA encoding the antigen of interest (50 ug, 50 μl×2 sites tibialis anterior) or with recombinant POXvirus (2×107 PFU). Sera were collected 3 weeks after each immunization, and assayed for parasite-specific antibody responses by indirect fluorescent antibody test (IFAT) against P. yoelii parasitized erythrocytes (FIG. 4) or antigen-specific antibodies by ELISA against PyHEP17 synthetic peptide or PyCSP recombinant protein (FIGS. 5a and 5b).

Methods: IFAT and ELISA Assays were Performed as Described in Example 1.

Data presented in FIG. 4 demonstrate antibody responses induced by one, two or three immunizations with PyHEP17 viral replicon particles (VRPs) by either subcutaneous (SQ) or intramuscular (IM) routes of administration, as measured in sera collected after one, two or three immunizations by indirect fluorescent antibody test (IFAT) against P. yoelii parasitized erythocytes. Also depicted are the response induced by two or three immunizations with control (GFP) VRPs.

Data presented in FIG. 5a demonstrate antibody responses induced by homologous immunization with PyCSP viral replicon particles (VRPs) by either subcutaneous (SQ) or intramuscular (IM) routes of administration or by heterologous immunization with VRP prime SQ/DNA boost or VRP prime SQ/POXvirus boost regimens, as measured in sera collected after one, two or three immunizations by ELISA against recombinant PyCSP capture antigen. Also depicted are the response induced by homologous immunization with plasmid DNA, heterologous immunization with DNA prime/POXvirus boost DNA, or homologous immunization with control DNA or control VRPs.

Data presented in FIG. 5b demonstrate antibody responses induced by homologous immunization with PyHEP17 VRPs by either subcutaneous (SQ) or intramuscular (IM) routes of administration or by heterologous immunization with VRP prime SQ/DNA boost or VRP prime SQ/POXvirus boost regimens, as measured in sera collected after one, two or three immunizations by ELISA against a synthetic peptide representing the immunodominant PyHEP17 B cell epitope as capture antigen. Also depicted are the response induced by homologous immunization with plasmid DNA, heterologous immunization with DNA prime/POXvirus boost DNA, or homologous immunization with control DNA or control VRPs.

Results represented in FIGS. 4, 5a, and 5b show that immune responses to a malaria antigen can be induced by a single immunization with VEE viral replicon particles expressing the antigen of interest but that the magnitude of the immune response can be enhanced by additional immunizing dose(s).

### Example 3

Immunogenicity of VRP in a Mouse Model of Malaria—Cellular Immune Activation

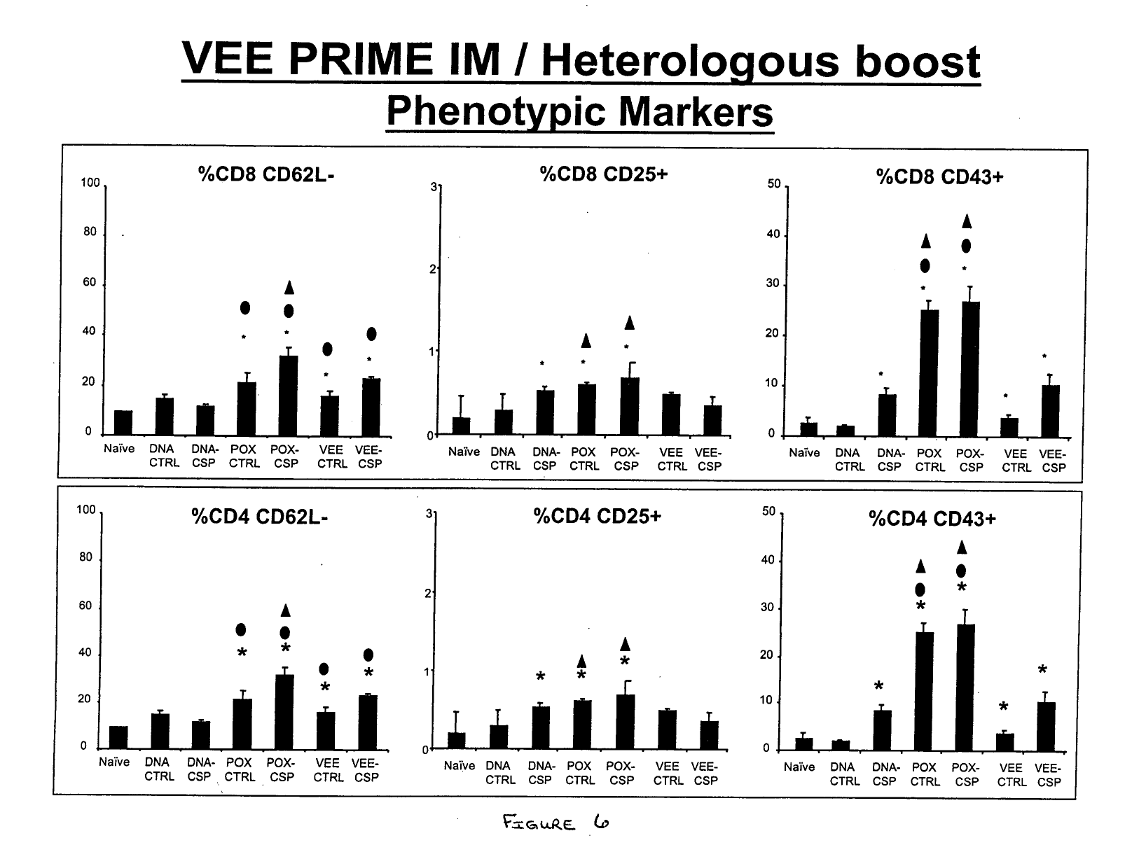

FIG. 6 demonstrates the expression of cell phenotypic and activation markers induced by PyCSP VRP in homologous or heterologous VRP prime IM vaccination strategies, as measured by multiparameter flow cytometry.

Study:

Female 4- to 8-wk-old AnNCr (H-2d) (cat# AnNCr) mice were obtained from The National Cancer Institute (Charles River Laboratories, Fredrick, Md.). In homologous VRP immunization regimens, groups of 6 mice were primed by IM immunization with X106 VEE viral replicon particles (VRP) encoding the malaria gene of interest (PyCSP or PyHEP17) and then boosted 6 weeks later with VEE viral replicon particles (VRPs) encoding the malaria gene of interest or an unrelated control gene (influenza HA). In VRP prime/heterologous boost immunization regimens, groups of 6 mice were primed by IM immunization with 1×10⁶ VEE viral replicon particles (VRPs) encoding the malaria gene of interest (PyCSP or PyHEP17) and then boosted 6 weeks later plasmid DNA (100 ug), recombinant poxvirus (1-2×10⁷ pfu) or recombinant adenovirus (1×10⁸ particles) encoding the same malaria gene. Phenotypic changes in bulk CD8+ or CD4⁺ T cell populations were evaluated at 3 days or 7 days following the boost.

Methods: Expression of cell phenotypic and activation markers induced by PyCSP VRP in homologous or heterologous VRP prime vaccination strategies was assayed by multiparameter flow cytometry essentialy as described in Example 1 except that cells were stained for cell surface markers (CD62L, CD69, CD25 CD43, CD71, CD152) rather than for intracellular cytokine production.

Data presented in FIG. 6 demonstrate the expression of phenotypic and activation markers induced by priming with PyCSP VRPs administered intramuscularly (IM) and homologous boosting with PyCSP VRPs or control VRP, or heterologous boosting (IM) with plasmid DNA encoding PyCSP or control DNA, or heterologous boosting (IM) with recombinant POXvirus expressing PyCSP or control POXvirus. Also depicted is the expression of phenotypic and activation markers in naive mice. Phenotypic changes in bulk CD8+ or CD4+ T cells were evaluated at 3 days or 7 days following boost. Responses at 7 days post immunization are presented, for CD62L (expressed by naïve T cells), CD25 (1L-2 receptor, expressed on activated & proliferating cells), CD43 (marker for activated CD8 CTL) expressed on gated CD8+ T cells. Not presented are data showing expression of CD69 (early activation marker), CD71 (transferin receptor, activation marker), and CD152 (CTLA-4, activation marker).

Results represented in FIG. 6 show that homologous and heterologous immunization with VEE viral replicon particles expressing malaria antigens can effectively activate both CD8+ and CD4+ compartments of the cellular immune system. In general, activation of CD8+ T cells by VRPs is more pronounced than for CD4+ T cells.

### Example 4

Immunogenicity of VRP prime/Heterologous Boost Immunization in a Mouse Model of Malaria

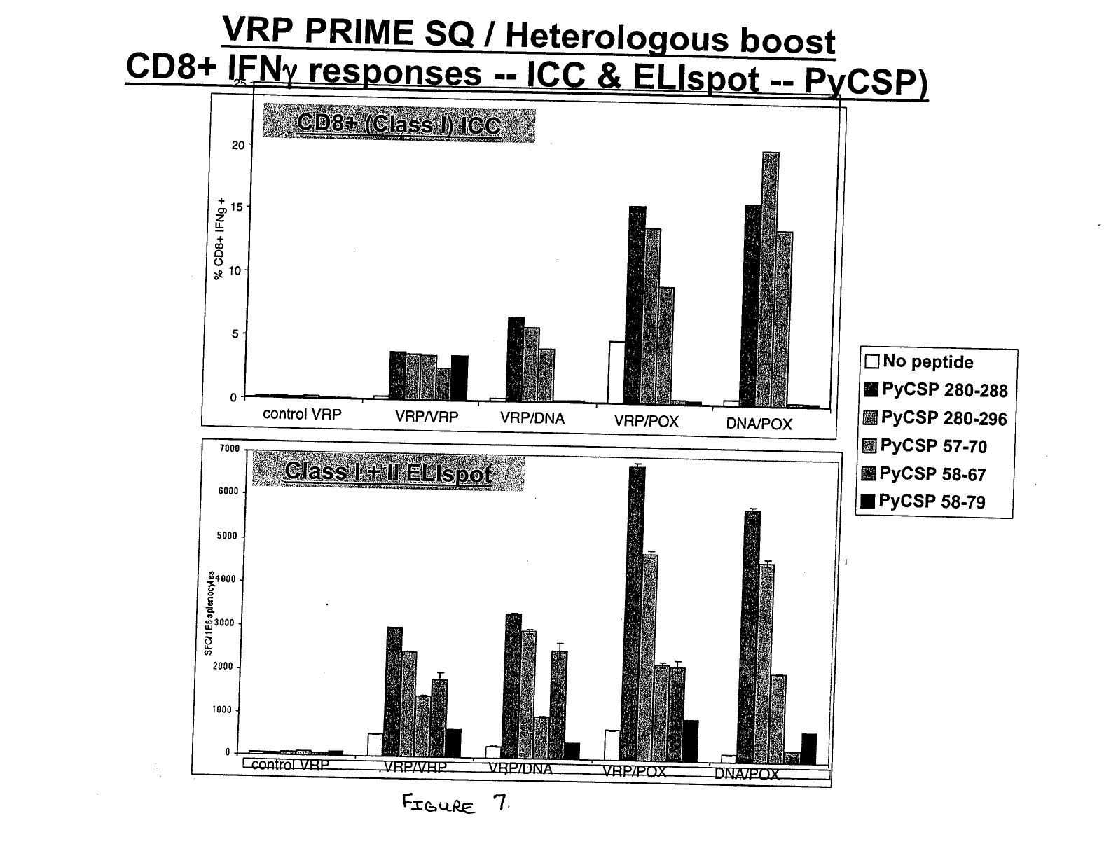

FIG. 7 demonstrates antigen-specific cytokine responses to PyCSP induced by PyCSP VRP using various routes of homologous immunization or by heterologous VRP prime SQ vaccination strategies, as measured by intracellular cytokine staining or ELIspot.

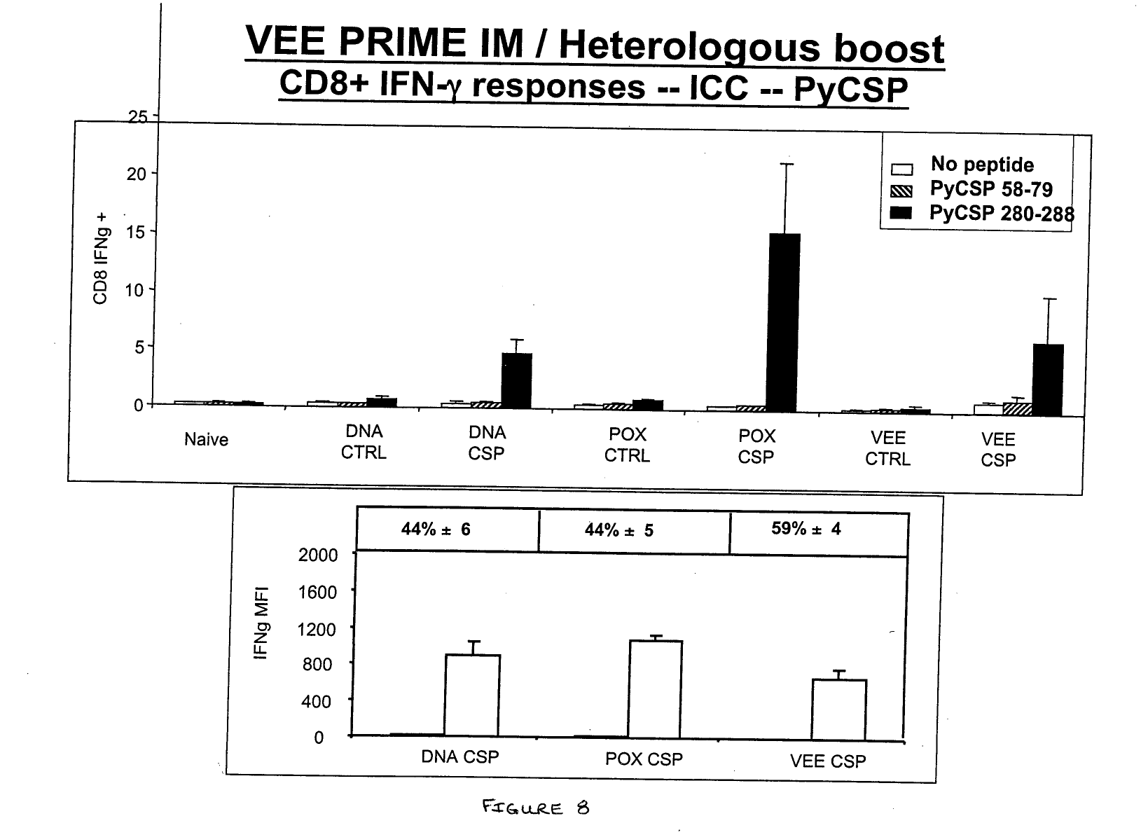

FIG. 8 demonstrates the frequency and magnitude of antigen-specific cytokine responses to PyCSP induced by PyCSP VRP in homologous IM or heterologous VRP prime IM vaccination strategies, as measured by intracellular cytokine staining.

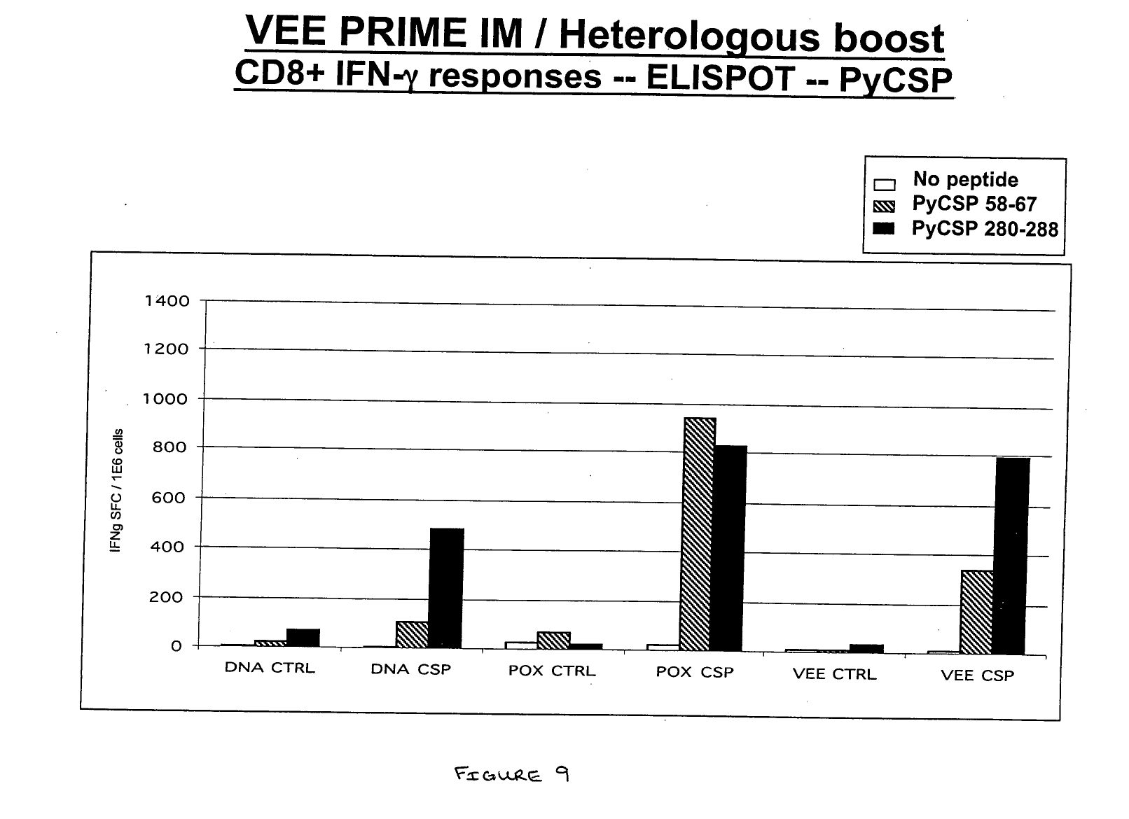

FIG. 9 demonstrates antigen-specific cytokine responses to PyCSP induced by PyCSP VRP in homologous IM or heterologous VRP prime IM vaccination strategies, as measured by ELIspot.

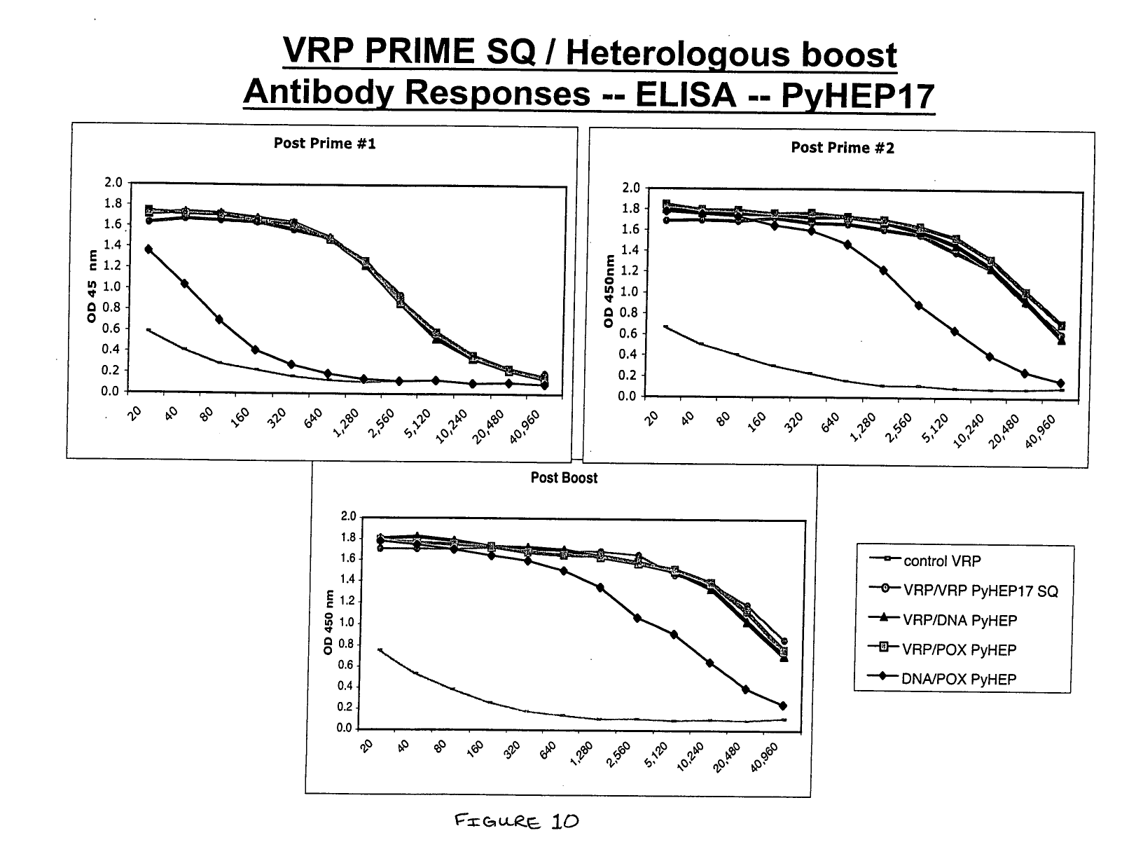

FIG. 10 demonstrates antigen-specific antibody responses to PyBEP17 induced by various doses of PyHEP17 VRP in homologous SQ or heterologous VRP prime SQ vaccination strategies, as measured by ELISA.

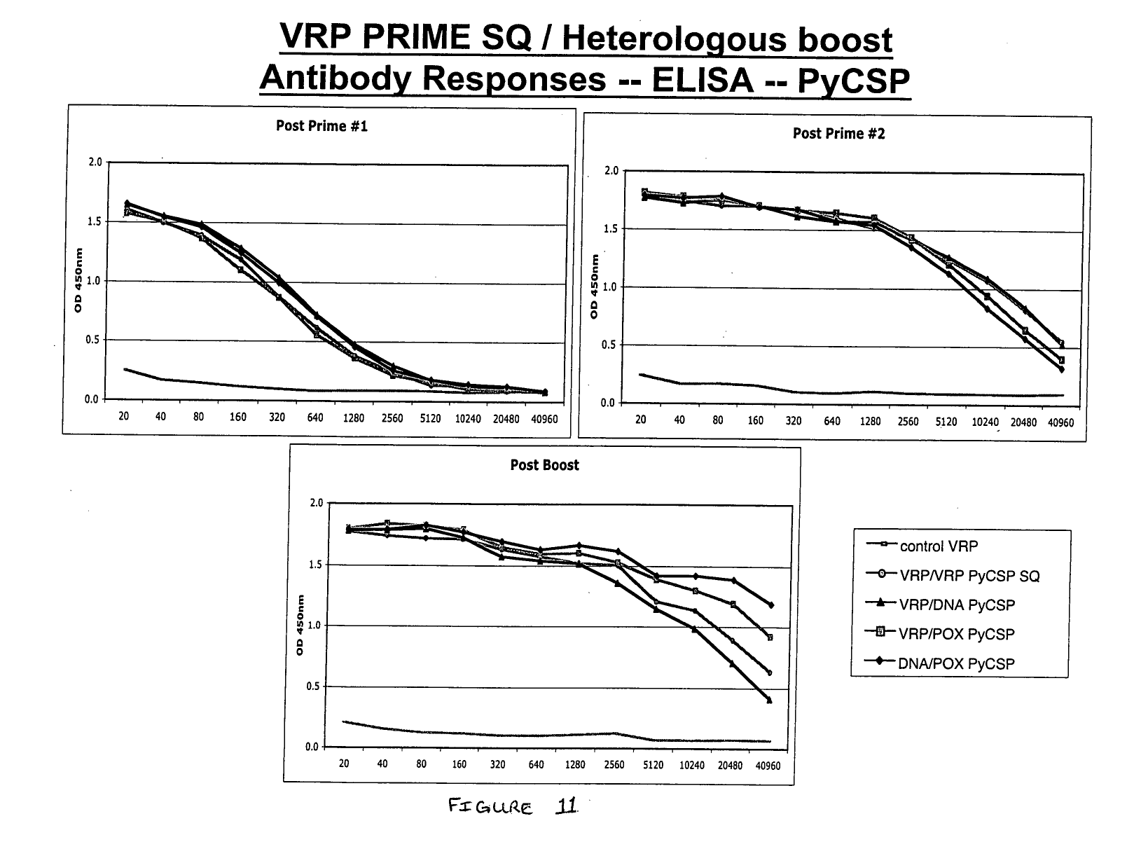

FIG. 11 demonstrates antigen-specific antibody responses to PyCSP induced by various doses by PyCSP VRP in homologous SQ or heterologous VRP prime SQ vaccination strategies, as measured by ELISA.

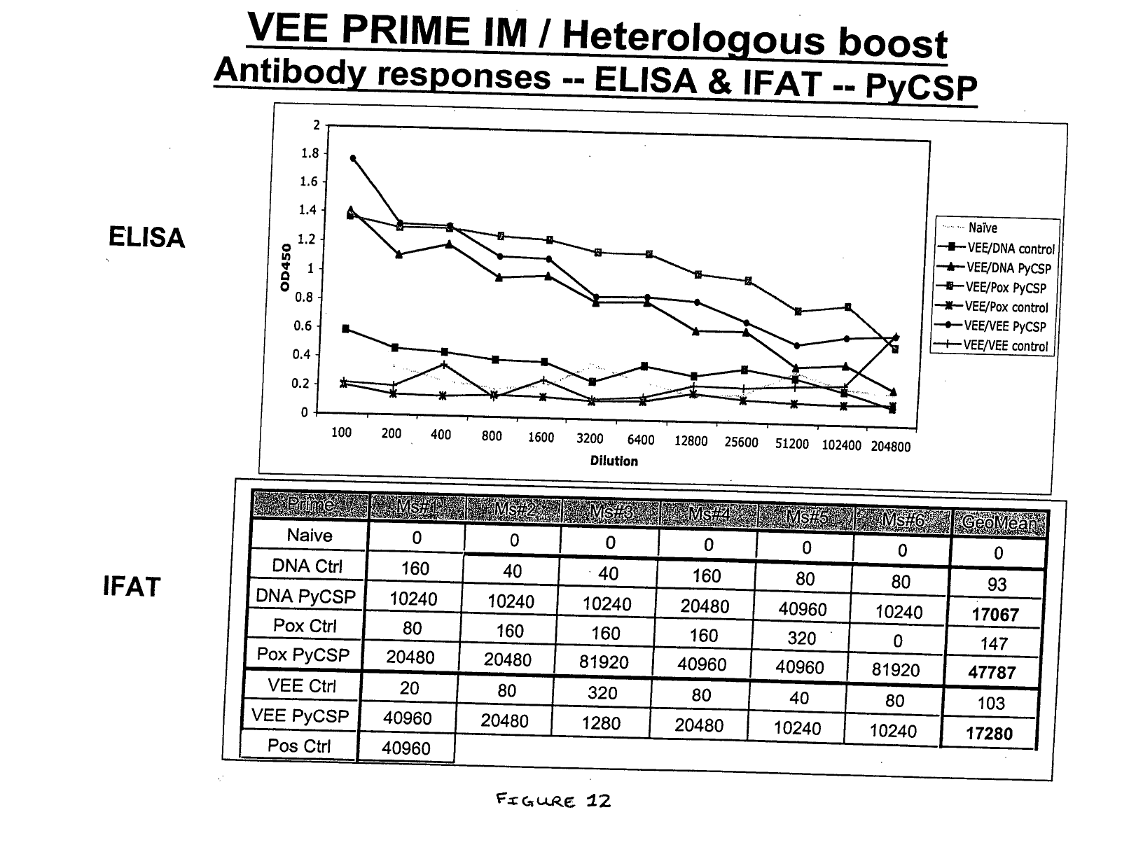

FIG. 12 demonstrates antigen-specific and parasite-specific antibody responses to PyCSP induced by various doses by PyCSP VRP in homologous IM or heterologous VRP prime IM vaccination strategies, as measured by ELISA against PyCSP antigen or indirect fluorescent antibody test (IFAT) against P. yoelii parasitized erythrocytes.

Study:

Female 4- to 8-wk-old BALB/cByJ (H-2d) (cat# JR001026) mice were obtained from The Jackson Laboratory, Bar Harbor, Me. Groups of 6 mice were primed by SQ immunization (footpad, 50 μl×2 sites) with 5×10⁶ VEE viral replicon particles (VRP) encoding the malaria gene of interest (PyHEP17 or PyCSP) or an unrelated control gene (GFP or HA) two times at 4 week intervals and then boosted IM 4 weeks later with VRP expressing the malaria gene of interest (homologous boosting) or with plasmid DNA encoding the antigen of interest (50 ug, 50 μl×2 sites, tibialis anterior) or with recombinant POXvirus (2×10⁷ PFU) (heterologous boosting). Splenocytes were harvested at 3 weeks after the boost, and assayed for antigen specific cell mediated immune responses by IFNg ELIspot or intracellular cytokine staining flow cytometry assays (FIG. 7). In addition, sera were collected 3 weeks after each immunization, and assayed for parasite-specific antibody responses by indirect fluorescent antibody test (IFAT) against P. yoelii parasitized erythrocytes (data not shown) or antigen-specific antibodies by ELISA against PyHEP17 synthetic peptide or PyCSP recombinant protein (FIG. 10 and 11).

In other studies, female 4- to 8-wk-old AnNCr (H-2d) (cat# AnNCr) mice were obtained from The National Cancer Institute (Charles River Laboratories, Fredrick, Md.). Groups of 6 mice were primed by IM immunization (tibialis anterior, 50 μl×2 sites) with 1×10⁶ VEE viral replicon particles (VRP) encoding the malaria gene of interest (PyCSP or PyHEP17) and then boosted IM 6 weeks later with VRP expressing the malaria gene of interest or an unrelated control gene (influenza HA) (homologous boosting) or with plasmid DNA encoding the antigen of interest or control DNA (50-100 ug, 50 μl×2 sites, tibialis anterior) or with recombinant POXvirus or control POXvirus (2×10⁷ PFU) (heterologous boosting). Splenocytes were harvested at 2 weeks after the boost, and assayed for antigen specific cell mediated immune responses by IFNg ELIspot or intracellular cytokine staining flow cytometry assays (FIGS. 8 and 9). In addition, sera were collected 3 weeks after each immunization, and assayed for parasite-specific antibody responses by indirect fluorescent antibody test (IFAT) against P. yoelii sporozoites (PyCSP) or parasitized erythrocytes (PyHEP17) or antigen-specific antibodies by ELISA against PyHEP17 synthetic peptide or PyCSP recombinant protein (FIG. 12).

Methods: Antibody assays (IFAT and ELISA) and cellular assays (IFNg ELIspot and intracellular cytokine staining flow cytometry) were performed as described in Example 1.

Data presented in FIG. 7 demonstrate antigen-specific cytokine (IFNg) responses induced by homologous immunization with PyCSP VRPs administered SQ or by priming with PyCSP VRPs administered SQ and heterologous boosting (IM) with plasmid DNA encoding PyCSP. Also depicted are the responses induced by homologous immunization with control VRPs or heterologous immunization with DNA prime/POXvirus boost. Splenocytes collected after three immunizations at 4 week inervals (two primes and one boost) were used as effector cells in ELIspot or intracellular cytokine flow cytometry (ICC, CD8+ gated population) assays with MHC-matched target cells expressing either class I (P815 cells) or class I and class II (A20.2J cells) pulsed with synthetic peptides representing defined PyCSP CD8+ and/or CD4⁺ T cell epitopes or without peptide. PyCSP residues 280-288=dominant CD8+ T cell epitope; PyCSP residues 280-295, overlapping dominant CD4+ and dominant CD8+ T cell epitope; PyCSP residues 57-70=dominant CD4+ T cell epitope; PyCSP residues 58-67=subdominant CD8+ T cell; and PyCSP residues 58-79=overlapping dominant CD4+ T cell epitope and subdominant CD8+ T cell epitope. Also depicted are the responses induced by homologous immunization with control (GFP) VRPs. ELIspot data are plotted as spot forming cells (SFC) per million splenocytes. ICC data are presented as the frequency of CD8+ T cells secreting IFNg.

Data presented in FIG. 8 demonstrate antigen-specific cytokine (IFNg) responses induced by priming with PyCSP VRPs administered IM and homologous boosting with PyCSP replicons or control replicon, or heterologous boosting (IM) with plasmid DNA encoding PyCSP or control DNA, or heterologous boosting (IM) with recombiannt POXvirus expressing PyCSP or control POXvirus. Splenocytes collected after two immunizations at 6 week intervals (one prime and one boost) were used as effector cells in intracellular cytokine staining assays (ICC, CD8+ gated population) for IFNg or TNFa with MHC-matched target cells expressing class I and class II (A20.2J cells) pulsed with synthetic peptides representing defined PyCSP CD8+ and/or CD4+ T cell epitopes or without peptide. PyCSP residues 280-288=dominant CD8+ T cell epitope; PyCSP residues 58-79=overlapping dominant CD4+ T cell epitope and subdominant CD8+ T cell epitope. ICC data are presented as the frequency of CD8+T cells secreting IFNg (% CD8+ IFNg+) or as the mean fluorescence intensity (IFNg MFI) for IFNg secreting CD8+ T cells. Also presented is the frequency of cells that secreted both IFNg and TFNa (double positive cells).

Data presented in FIG. 9 demonstrate antigen-specific cytokine (IFNg) responses induced by priming with PyCSP replicons administered IM and homologous boosting with PyCSP replicons or control replicon, or heterologous boosting (IM) with plasmid DNA encoding PyCSP or control DNA, or heterologous boosting (IM) with recombinant POXvirus expressing PyCSP or control POXvirus. Splenocytes collected after two immunizations at 6 week intervals (one prime and one boost) were used as effector cells in IFNg ELIspot assays with MHC-matched target cells expressing class I and class II (A20.2J cells) pulsed with synthetic peptides representing defined PyCSP CD8+ and/or CD4+ T cell epitopes or without peptide. PyCSP residues 280-288=dominant CD8+ T cell epitope; PyCSP residues 58-79=overlapping dominant CD4+ T cell epitope and subdominant CD8+ T cell epitope. ELIspot data are plotted as spot forming cells (SFC) per million splenocytes.

Data presented in FIG. 10 demonstrate antigen-specific antibody responses induced by priming with PyHEP17 replicons administered SQ and homologous boosting with PyHEP17 replicons, or heterologous boosting (IM) with plasmid DNA encoding PyHEP17, or heterologous boosting (IM) with recombinant POXvirus expressing PyHEP17. Also depicted are the responses induced by homologous immunization with control replicons or heterologous immunization with DNA prime/POXvirus boost. Sera collected after one, two, or three immunizations at 4 week intervals (two primes and one boost) were assayed by ELISA against a synthetic peptide representing the immunodominant PyHEP17 B cell epitope as capture antigen.

Data presented in FIG. 11 demonstrate antigen-specific antibody responses induced by priming with PyCSP replicons administered SQ and homologous boosting with PyCSP replicons, or heterologous boosting (IM) with plasmid DNA encoding PyCSP, or heterologous boosting (IM) with recombinant POXvirus expressing PyCSP. Also depicted are the responses induced by homologous immunization with control replicons or heterologous immunization with DNA prime/POXvirus boost. Sera collected after one, two, or three immunizations at 4 week intervals (two primes and one boost) were assayed by ELISA against recombinant PyCSP protein as capture antigen.

Data presented in FIG. 12 demonstrate antigen-specific antibody responses induced by priming with PyCSP replicons administered IM and then boosted (homologous boosting) with PyCSP replicons or control replicons, or boosted (heterologous boosting) with plasmid DNA encoding PyCSP or control DNA, or with recombinant POXvirus expressing PyCSP or control POXvirus. Sera collected after two immunizations at 6 week intervals (one prime and one boost) were assayed by ELISA against a synthetic peptide representing the immunodominant PyCSP B cell epitope as capture antigen, or by IFAT against P. yoelii sporozoites.

Results represented in FIGS. 7-12 show that heterologous prime/boost immunization strategies comprising priming with VEE viral replicon particles expressing malaria antigens and heterologous boosting with non-alphavirus expression systems can effectively induce antigen-specific and parasite specific antibody responses as well as cell mediated immune responses. VRP expressing malaria antigens can effectively prime for induction of immune responses against both immunodominant and subdominant CD8+ and CD4+ T cell epitopes, thereby achieving a profile of broad epitope recognition and broad immune response in the vaccinated population.

### Example 5

Protective Efficacy of Homologous VRP Immunization and VRP Prime/Heterologous Boost Immunization in a mouse model of malaria

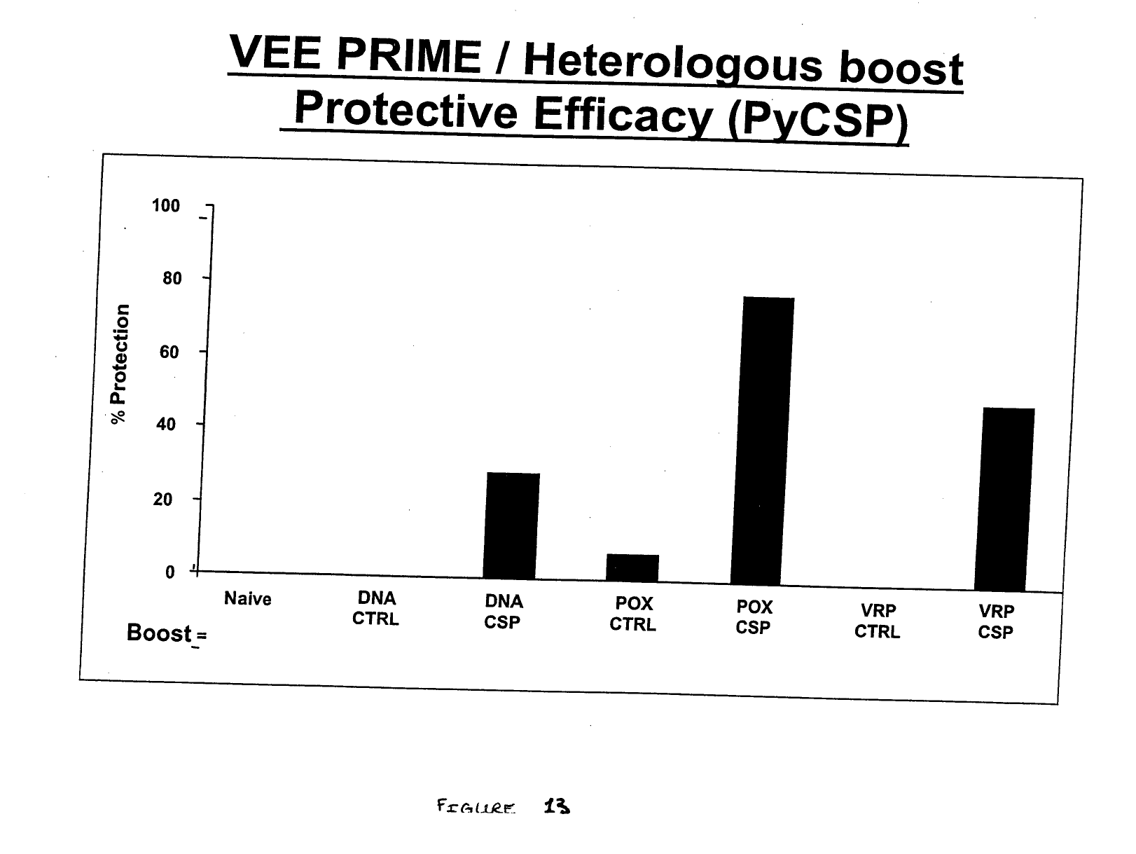

FIG. 13 demonstrates the capacity of homologous IM or heterologous VRP prime IM vaccination strategies with PyCSP VRP to protect against P. yoelii parasite challenge.

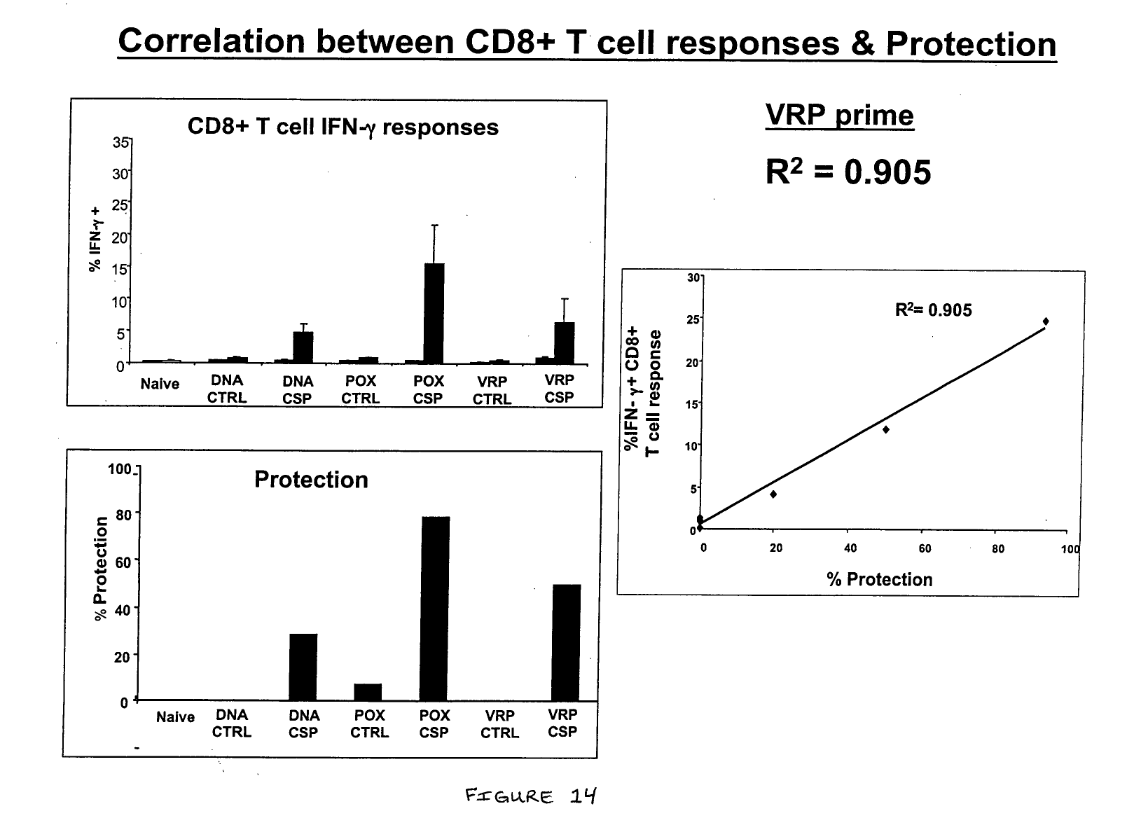

FIG. 14 demonstrates the correlation between CD8+ T cell IFNg responses induced by homologous or heterologous VRP prime IM vaccination strategies and protection against P. yoelii parasite challenge.

Study:

Female 4- to 8-wk-old AnNCr (H-2d) (cat# AnNCr) mice were obtained from The National Cancer Institute (Charles River Laboratories, Fredrick, Md.). Groups of 12 mice were primed by IM immunization (tibialis anterior, 50 μl×2 sites) with 1×10⁶ VEE viral replicon particles (VRP) encoding the malaria gene of interest (PyCSP) and then boosted IM 6 weeks later with VRP expressing PyCSP or an unrelated control gene (influenza HA) (homologous boosting) or with plasmid DNA encoding PyCSP or control DNA (50 ug, 50 μl×2 sites, tibialis anterior) or with recombinant POXvirus expressing PyCSP or control POXvirus (1×10⁷ PFU) (heterologous boosting). Mice were challenged with infectious P. yoelli sporozoites (100 sporozoites) at 2 weeks after the boost, and followed for 14 days for the presence or absence of blood-stage parasitemia.

Methods:

Blood stage protection against challenge with Plasmodium yoelii parasites: P. yoelii (17XNL nonlethal strain, clone 1.1) parasite was maintained by alternating passage of the parasites in Anopheles stephensi mosquitoes and CD-1 mice. Sporozoites were harvested from nonirradiated P. yoelii 17×NL infected mosquitoes 14 days after an infectious blood meal by hand-dissection. Mice (n=12 per group) were challenged by tail-vein injection of 100 infectious sporozoites in a 0.2 ml volume of M199 containing 5% normal mouse serum. Since it has been established previously that infection with as few as one or two sporozoites of P. yoelii 17×NL will result in patent infection of 50% of BALB/c mice (ID50), the challenge dose used here represents a virulent paarasite challenge. Giemsa-stained thin blood films were examined on days 5-14 post-challenge, up to 50 oil-immersion fields being examined for parasites. Protection was defined as the complete absence of blood-stage parasitemia.

Data presented in FIG. 13 demonstrate the percentage of mice that were completely protected against development of blood-stage parasitemia (sterile protection) following challenge with infectious P. yoelii sporozoites.

Data presented in FIG. 14 demonstrate the correlation between CD8+IFNg responses induced by priming with PyCSP replicons and boosting with PyCSP replicons, plasmid DNA encoding PyCSP, or recombinant POXvirus expressing PyCSP, and protection against P. yoelii parasite challenge as determined by simple linear regression analysis.

Results represented in FIGS. 13 and 14 show that homologous VRP immunization or heterologous primetboost immunization strategies comprising priming with VEE viral replicon particles expressing malaria antigens and heterologous boosting with non-alphavirus expression systems can effectively induce sterile protective immunity against malaria parasite challenge.

Synthetic Peptides

Synthetic peptides based on PyCSP or PyHEP17 sequences used for in vitro stimulation for T cell assays were synthesized commercially at >90% purity (AnaSpec Inc., San Jose, Calif.; Research Genetics, Huntsville, Ala.)

### Example 6

Construction of VRPs

A. Construction of Replicon Plasmids Encoding Malarial Genes

The single-promoter vector plasmid, pERK, is a derivative of pVR21 [Pushko et al., 1997] that has been modified to contain an expanded multiple-cloning site, remove the ampicillin resistance gene, and add a kanamycin resistance gene as the selectable marker. The pERK plasmid was used to engineer the single-promoter replicon RNAs expressing PyCSP, PyHEP17, PkCSP, PkMSP1-42, PkAMA1, PkSSP2, PFEBA FVO and PfMSP1-42 FVO.

B. Production of VRPs

RNA transcripts are produced in vitro (Promega RiboMAX transcription kits) from the two VEE structural protein gene helper plasmids (capsid and glycoprotein) and the VEE replicon vector plasmid encoding the malarial antigen. The three RNA transcript preparations are separately purified by either spin-column (gel binding and elution) or size-exclusion chromatography, followed by agarose gel analysis to assess integrity, and quantitated by UV absorbance. The three RNA preparations are combined with Vero cells in a 1 mL electroporator cuvette and pulsed four times with a device set to deliver 580 volts at 25 μF. After electroporation the cells were incubated at room temperature for 10 minutes and seeded into flasks containing serum-free medium. The cultures were incubated at 37° C. in 5% CO₂ for 18-24 hours. VRPs were collected from transfected cells and concentrated and purified by binding to, and elution from, pre-packed, Heparin Fast Flow columns (Pharmacia).

TABLE 1: Synthetic Peptide Sequences

IA. PyCSP Peptides

PyCSP residues (280-295) S Y V P S A E Q I L E F V K Q I (SEQ ID NO.: 1) Overlapping dominant CD4⁺ and dominant CD8⁺ T cell epitope
PyCSP residues (280-288) S Y V P S A E Q I (SEQ ID NO.:2) Dominant CD8⁺ T cell epitope
PyCSP (58-79) Y N R N I V N R L L G D A L N G K P E E K (SEQ ID NO. 3) overlapping dominant CD4+ T cell epitope and subdominant CD8+T cell epitope
PyCSP (58-67) I Y N R N I V N R L (SEQ ID NO.:4) Subdominant CTL, subdominant CD8+ T cell epitope
PyCSP (57-70) K I Y N R N I V N R L L G D (SEQ ID NO.:5) CD4+ T cell LPA epitope, Dominant CD4⁺ T cell epitope
   IB. PyHEP17 Peptides
15-mer (nested CD8+T cell epitopes) and 9-mer CTL epitopes:
PyHEP₆₁₋₇₅ #4612 (EEIVKLTKNKKSLRK) Dominant CD4+ T cell epitope (SEQ ID NO.:6)
PYHEP₆₆₋₈₀ #4613 (LTKNKKSLRKINVAL) Subdominant CD4+ T cell epitope (SEQ ID NO.:7)
PyHEP₇₁₋₈₅ #4614 (KSLRKINVALATAL) Dominant CD4+ T cell epitope (SEQ ID NO.: 8)
PYHEP₇₃₋₈₁ (LRKINVALA) Ssubdominant CD8+T cell epitope (SEQ ID NO.:9)
PyHEP₇₄₋₈₂ (RKINVALAT) Ssubdominant CD8+ T cell epitope (SEQ ID NO.: 10) 15-mer LPA epitopes:
PyHEP₉₆₋₁₁₀ #4619 (GLVMYNTEKGRRPFQ) Subdominant CD4+ T cell epitope (SEQ ID NO.: 11)
PyHEP₁₂₆₋₁₄₀ #4625 (SFPMNEESPLGFSPE) Subdominant CD4+ T cell epitope, Dominant B Cell epitope (SEQ ID NO.: 12)
PYHEP₁₃₆₋₁₅₀ 190 4627 (GFSPEEMEAVASKFR) Subdominant CD4+ T cell epitope (SEQ ID NO.: 13)
   IC. PyCSP Repeat Peptide

1. PyCSP repeat, sequence (QGPGAPQGPGAPQGPGAP) Dominant B Cell Epitope (SEQ ID NO.: 14)

Obviously, many modifications and variations of the present invention are possible in light of the above teachings. It is therefore to be understood that, within the scope of the appended claims, the invention may be practiced otherwise than as specifically described.

1. A method to immunize a subject against malarial disease comprising:
a. administering to the subject a priming immunization preparation comprising one or more alphavirus replicons expressing a gene encoding a malarial antigen or combination of malarial antigens; and
b. subsequently administering to the subject a boosting immunization preparation comprising the malarial antigen or combination of malarial antigens, said preparation being selected from the group consisting of
1) a recombinant non-alphavirus viral expression system encoding the malarial antigen;
2) a preparation of the malarial protein antigen produced by recombinant DNA technology;
3) a synthetic preparation of the malarial antigen;
4) a malarial organism or extract thereof; and
5) a polynucleotide vector expressing the malarial antigen, or a combination thereof.

2. The method of claim 1 wherein the alphavirus replicon preparation is selected from the group consisting of RNA replicons, DNA replicons, and alphavirus replicon particles.

3. The method of claim 2, wherein the alphavirus is selected from the group consisting of Venuezuelan Equine Encephalitis Virus, Semliki Forest Virus, and Sindbis Virus.

4. The method of claim 1, wherein the malarial antigen is selected from the group consisting of a full-length malarial antigen, an immunogenic fragment thereof, or an epitope derived from the malarial antigen, or a combination thereof.

5. The method of claim 4, wherein the malarial antigen is selected from the group of malarial pathogens consisting of Plasmodium falciparum, Plasmodium vivax, and Plasmodium ovale.

6. The method of claim 5, wherein the malarial antigen is expressed at a stage of the malarial parasite life cycle selected from the group consisting of preerythrocytic, erythrocytic and transmission blocking.

7. The method claim 6, wherein the malarial antigen is selected from the group consisting of: PfCSP, PFEXP1, PfSSP2, PfLSA-1, PfLSA-3, PfMSP-1, PfAMA-1, PfEBA-175, PfMSP-3, PfMSP-4, PfMSP-5, PfRAP-1, PfRAP-2.

8. The method of claim 1, wherein the non-alphavirus viral expression system is selected from the group consisting of poxvirus, adenovirus, adenoassociated virus, and retrovirus.

9. The method of claim 8, wherein the poxvirus is selected from the group consisting of cowpox, canarypox, vaccinia, modified vaccinia Ankara, or fowlpox.

10. The method of claim 1 wherein the malarial antigen is selected from the group of malarial parasites consisting of Plasmodium falciparum, Plasmodium vivax, and Plasmodium ovale.

11. The method of claim 1, wherein multiple boosting immunization doses are administered.

12. The method of claim 2, wherein the alphavirus replicon is a naked nucleic acid and the priming immunization preparation consists of 1, 2, 3, or 4 doses of the naked nucleic acid.

13. The method of claim 1, wherein the priming immunization preparation is administered by a route selected from the group consisting of: subcutaneously, intramuscularly, intradermally, mucosally, orally, and by specialized injection devices.

14. The method of claim 1, wherein the boosting immunization preparation is administered by a route selected from the group consisting of: subcutaneously, intramuscularly, intradermally, mucosally, orally, transcutaneously, and by specialized injection devices.

15. The method of claim 13 or 14 wherein the priming and boosting immunization preparations are administered by the same route.

16. The method of claim 13 or 14 wherein the priming and boosting immunization preparations are each administered by a different route.

17. A method to immunize a subject against malarial disease comprising:
a. administering to the subject a priming immunization preparation comprising Venezuelan Equine Encephalitis replicon particles expressing a gene encoding a malarial antigen, wherein said malarial antigen is selected from the group consisting of a full-length malarial antigen, an immunogenic fragment thereof, and an epitope derived from the malarial antigen; and
b. subsequently administering to the subject a boosting immunization preparation comprising the malarial antigen, said preparation comprising a poxvirus encoding the malarial antigen.

18. An immunogenic composition comprising two immunizing components, wherein the first immunizing component comprises alphavirus replicons expressing a gene encoding a malarial antigen, and wherein the second immunizing component comprises a preparation expressing the malarial antigen, said preparation being selected from the group consisting of 1) a recombinant non-alphavirus viral expression system encoding the malarial antigen;
2) a preparation of the malarial protein antigen produced by recombinant DNA technology;
3) a synthetic preparation of the malarial antigen;
4) a malarial organism or extract thereof; and
5) a polynucleotide vector expressing the malarial antigen,
or a combination thereof and wherein said malarial antigen is selected from the group consisting of a full-length malarial antigen, an immunogenic fragment thereof, and an epitope derived from the malarial antigen.

19. The immunogenic composition of claim 18, wherein said first immunizing component, said second immunizing component or both further comprise an adjuvant.

20. The immunogenic composition of claim 19 in combination with a pharmaceutically acceptable carrier.

21. An immunogenic composition comprising two immunizing components, wherein the first immunizing component comprises alphavirus replicon particles expressing a gene encoding a malarial antigen, and wherein the second immunizing component comprises a poxvirus vector expressing the malarial antigen.

22. The immunogenic composition of claim 21 wherein the alphavirus replicon particle is derived from VEE.

23. An immunogenic composition comprising two immunizing components, wherein the first immunizing component comprises alphavirus replicon particles expressing a gene encoding a malarial antigen, and wherein the second immunizing component comprises a adenovirus vector expressing the malarial antigen.

24. The immunogenic composition of claim 21, wherein the alphavirus replicon particle is derived from VEE.

25. An immunogenic composition comprising two immunizing components, wherein the first immunizing component comprises alphavirus replicon particles expressing a gene encoding a malarial antigen, and wherein the second immunizing component comprises a plasmid DNA construct expressing the malarial antigen.

26. The immunogenic composition of claim 21, wherein the alphavirus replicon particle is derived from VEE.


SEQUENCE LISTING (14 sequences)

SEQ ID NO: 1 - protein, 16 aa, Plasmodium yoelii
SYVPSAEQILEFVKQI

SEQ ID NO: 2 - protein, 9 aa, Plasmodium yoelii
SYVPSAEQI

SEQ ID NO: 3 - protein, 21 aa, Plasmodium yoelii
YNRNIVNRLLGDALNGKPEEK

SEQ ID NO: 4 - protein, 10 aa, Plasmodium yoelii
IYNRNIVNRL

SEQ ID NO: 5 - protein, 14 aa, Plasmodium yoelii
KIYNRNIVNRLLGD

SEQ ID NO: 6 - protein, 15 aa, Plasmodium yoelii
EEIVKLTKNKKSLRK

SEQ ID NO: 7 - protein, 15 aa, Plasmodium yoelii
LTKNKKSRLKINVAL

SEQ ID NO: 8 - protein, 14 aa, Plasmodium yoelii
KSRLKINVALATAL

SEQ ID NO: 9 - protein, 9 aa, Plasmodium yoelii
LRKINVALA

SEQ ID NO: 10 - protein, 9 aa, Plasmodium yoelii
RKINVALAT

SEQ ID NO: 11 - protein, 15 aa, Plasmodium yoelii
GLVMYNTEKGRRPFQ

SEQ ID NO: 12 - protein, 15 aa, Plasmodium yoelii
SFPMNEESPLGFSPE

SEQ ID NO: 13 - protein, 15 aa, Plasmodium yoelii
GFSPEEMEAVASKFR

SEQ ID NO: 14 - protein, 18 aa, Plasmodium yoelii
QGPGAPQGPGAPQGPGAP